(19) United States
(12) Patent Application Publication
(10) Pub. No.: US 2023/0263573 A1
(43) Pub. Date: Aug. 24, 2023

(54) PROBES, SYSTEMS, AND METHODS FOR COMPUTER-ASSISTED LANDMARK OR FIDUCIAL PLACEMENT IN MEDICAL IMAGES

(71) Applicant: KALIBER LABS INC., San Francisco, CA (US)

(72) Inventors: Tim BAKHISHEV, San Francisco, CA (US); Chandra JONELAGADDA, San Francisco, CA (US); Mark RUIZ, San Francisco, CA (US); Ryan BARBAN, San Francisco, CA (US); Ray RAHMAN, San Francisco, CA (US)

(21) Appl. No.: 18/003,311

(22) PCT Filed: Jun. 25, 2021
(86) PCT No.: PCT/US21/39200

§ 371 Date: Dec. 23, 2022

Related U.S. Application Data
(60) Provisional application No. 63/044,011, filed on Jun. 25, 2020.

(51) Int. Cl. A61B 34/20; G06T 7/12; G06T 11/60; G06T 9/00

(52) U.S. Cl. CPC A61B 34/20; G06T 7/12; G06T 11/60; G06T 9/00; G06T 2207/20081; G06T 2207/10068; G06T 2207/30008

(57) ABSTRACT

Various embodiments provide probes, systems and methods to assist an arthroscopic, endoscopic or other surgical procedure by the placement of digital landmarks in selected locations in a surgical field of view. Many embodiments utilize a specialized probe having a tip with a spherical or other selected geometry of known dimensions to assist with placement of the landmark. The probe is placed at a desired anatomical location and imaged by an arthroscopic/endoscopic video camera. Embodiments may use a deep learning network to analyze the image data encoded by the camera, identify the probe and generate a segmented outline. Then, computer vision/shape fitting algorithms are used to generate a refined outline of the probe wherein the dimensions of the probe tip are used to improve the accuracy of the image including the tip. The improved tip image accuracy in turn improves the accuracy of the placement of a landmark using the tip.

### CROSS-REFERENCE TO RELATED APPLICATIONS

This PCT application claims the benefit of U.S. Provisional Patent Application No. 63/044,011, filed Jun. 25, 2020, the entire contents of which are fully incorporated herein by reference for all purposes.

### BACKGROUND

Field of the Invention: Embodiments of the invention relate to systems, devices, and methods to guide and assist and surgical procedures, more particularly those using Artificial Intelligence (AI) to perform measurements of tissue and landmarking of selected anatomical/tissue sites in the course of a minimally invasive or other surgical procedure and still more particularly, probes or other surgical tools used in conjunction with AI algorithms to perform measurements and placement of digital landmarks at a target anatomical/tissue site during the course of a minimally invasive or other surgical procedure.

In recent years, Artificial Intelligence (AI) has begun to be used to assist with surgeries. However, current AI-assisted surgical systems and methods have drawbacks. For example, during the course of an arthroscopic or other minimally invasive procedure, surgeons use physical probes or other surgical tools to assist in the placement of physical markings or digital landmarks at a target tissue site. However, there is often uncertainty in the determination of the intended location due to imprecision in recognizing and/or sizing of the probe. Such imprecision can be due in part to changing viewing angles of the probe and/or partial obstruction of the probe. Thus, there is need for improved probes and related AI based surgical systems and methods for placement of digital landmarks.

### SUMMARY

Various embodiments described herein relate to computer-implemented medical systems, devices, and methods to guide a surgical procedure, for example by identifying and labelling anatomical features in real-time and placing one or more virtual (e.g., digital) landmarks on the identified anatomical feature. Many embodiments do so with the use of specialized probes or tools to improve the accuracy and precision of virtual landmark placement and subsequent measurements based on the landmarks.

Surgeons use physical landmarks to underpin a variety of cognitive tasks, for example, keeping track of latent vascularity, staple lines, suture locations, latent or otherwise hidden anatomical structures, etc. The landmarks can be placed using dyes, cauterization marks, etc. In some embodiments, needles are inserted from the outside to mark points. Placing a physical landmark which may require implanting an object in the patient's body may add to complications of the surgery and physically inhibit movement of surgical tools in the course of surgery. Other issues may involve a mistake made by an operator in the course of a surgery which can be costly. For example, for an operator to know the exact location of a critical anatomical feature that is hidden from a camera (e.g., a camera used during an arthroscopic or endoscopic surgery) may be difficult or impossible. For the operator to identify the location of a landmark after a change of field of view may also be difficult. Therefore, computer-implemented medical systems, devices, and methods such as Artificial Intelligence (AI) tools, particularly those for guiding medical procedures by applying a virtual landmark (e.g., a digital landmark) on the patient's body (e.g., on an organ or anatomical feature) can be valuable. These AI tools can be limited in accurately and reliably predicting a tool, anatomical structure, or detecting a procedure. In a fast-paced surgical procedure, the AI tool may need to also make predictions with low latency to provide real-time assistance to an operator.

Recognized herein is the need for fast, accurate, and reliable AI tools to assist an operator in real-time during the course of a surgical operation or other medical procedure by placing a virtual landmark on a location of interest to facilitate the surgical or other medical procedure for the operator (e.g., surgeon, interventional radiologist) and to improve an outcome of the surgery or other medical procedure. Accordingly, various aspects and embodiments described herein provide a pipeline of machine learning algorithms that is versatile and trained for unique needs of landmarks in various medical procedures. In many embodiments, one or more of the machine learning algorithms in the pipeline are configured to make use of a specialized probe or other surgical tool to facilitate placement of a digital landmark (e.g., a colored dot such as a blue dot, also referred to herein by the term “BluDot”) or fiducial described herein. In specific embodiments, specialized probes are employed which a tip or other portion having a shape, pattern, or texture, which can be readably identified and accurately measured (e.g., in width, area, or other dimension) from multiple viewing angles and/or lighting conditions by machine learning algorithms and/or deep learning networks. The ease and accuracy of identification of such probes in turn allows for the more accurate placement of a digital landmark allowing for more accuracy in various measurement or other surgical operations based on the landmarks such as the placement of sutures, surgical screws (or other anchors) and implants, and/or the sizing of implants.

Various embodiments described herein provide systems, devices, and methods that can receive information (e.g., image, voice, user inputs) prior to and during a medical procedure (e.g., a surgery, including a minimally invasive surgery), process the received information to identify features associated with placing a landmark associated with the procedure, and place a virtual landmark at a location of interest in real-time during the procedure. Many embodiments can do so with the use of a specialized probe or tool with a tip having selected shape and target point, which facilitates placement of the landmark. Particular embodiments of the probe have a spherical or other related shape whose recognition by AI and/or machine vision algorithms is relatively unaffected by the viewing angle of an arthroscope or other medical imaging device used during the surgery.

Systems, devices, and methods described herein can also aid surgeons to place a landmark on a location of interest intraoperatively by using images acquired preoperatively using imaging modalities and associated methods, such as fluoroscopy, magnetic resonance imaging (MRI), or computed tomography (CT) scanning. In one or more embodiments, the preoperative images can be that of a surgical field of view and Artificial Intelligence (AI) can be applied to preoperatively generated images to overlay the images and/or location of a landmark onto a real-time video stream of a surgical procedure to provide guidance to a surgeon. AI modules/algorithms can be used intraoperatively, preoperatively, or postoperatively to assist with the surgical procedure or improve an outcome of the procedure as Surgical AI.

Further provided herein are systems for assisting in minimally invasive procedures, such as arthroscopic procedures (e.g., to repair a shoulder, knee, hip, ankle, or other joint) by allowing computer-implemented arbitrary landmark placement (e.g., a digital landmark), the system comprising one or more computer processors and one or more non-transitory computer-readable storage media storing instructions that are operable, when executed by the one or more computer processors, to cause the one or more computer processors to perform operations comprising: receiving a video stream from an arthroscopic imaging device; receiving one or more sets of coordinates of one or more landmarks; overlaying the one or more landmarks on the video stream; and displaying the overlay on one or more display devices intraoperatively to be used by an operator during the arthroscopic procedure. Application of embodiments of the system to the assistance to other medical procedures (e.g., by the placement of arbitrary landmarks) including minimally invasive procedures, such as endoscopic, laparoscopic, and interventional cardiovascular procedures is also contemplated. Examples of such minimally invasive procedures can include one or more of gastro-intestinal (GI) procedures (e.g., biopsy of the intestines, removal of polyps, bariatric surgery, stomach stapling/vertical banded gastroplasty), urological procedures (e.g., removal of kidney stone, bladder repair), gynecological procedures (e.g., a dnc, removal of uterine fibroids), laparoscopic procedures (e.g., an appendectomy, cholecystectomy, colectomy, hernia repair, nissen fundoplication), and interventional cardiovascular procedures (e.g., heart valve replacement, coronary artery bypass surgery, vascular graft surgery, coronary or peripheral angioplasty, stenting of a coronary or other artery, or cardiac ablation for treatment of atrial fibrillation or other cardiac rhythm disorder).

According to one embodiment, provided herein is a system for performing tissue landmarking during a minimally invasive medical procedure such as an arthroscopic procedure where the landmarking performed with the aid of a surgical tool having a tip of a selected/known geometry and size. The system comprises one or more processors which may correspond to a central processing unit, a graphical processing unit or an application specific integrated circuit. The system can also comprise a memory to store instructions operable on the processors and an interface to receive a video stream encoding imaging data from a medical imaging device positioned to image a scene including the tool, e.g., when the tool is positioned at a selected tissue location of a patient. The one or more processors can execute instructions stored in the memory to process image data received from the interface and to perform operations comprising: recognizing the tool from the image data and generating a segmented tool outline depicting just the pixels where the tool is detected, the segmented tool outline represented as a pixel mask; fitting a shape of the tool onto the pixel mask utilizing the selected geometry and size of the tool tip, where the fitted shape minimizes error or inaccuracy in a shape of the pixel mask, mapping an intended location of the landmark based on the geometry and size of the tool tip, utilizing the fitted tool shape and mapped location to generate a digital landmark at a selected tissue location where the tool tip is positioned; and overlying the digital landmark onto the video stream so that it may be displayed on one or more screens or other display devices. The scene can be that of a selected tissue site or intraoperative tissue site. The imaging device can be positioned inside the patient's body but alternatively, can be positioned externally as well depending upon the procedure and the imaging modality. The error or inaccuracy of the pixel mask can correspond to the tip. The type of error or inaccuracy that is corrected for by the system can be caused by a viewing angle of the imaging device relative to the tool or the tool tip, or suboptimal lighting of the tool and/or the tool tip. In one or more embodiments, the mapping of the intended location of the landmark is made based on a predetermined target point of the probe tip which may be determined using a machine learning algorithm dataset and/or training data set. The medical imaging device may correspond to an arthroscope, an endoscope, a laparoscope, a cardioscope, or other minimally invasive medical imaging device. In these and related embodiments, the minimally invasive procedure can be an orthopedic procedure, an arthroscopic procedure, a shoulder procedure, a knee procedure, an endoscopic procedure, a laparoscopic procedure, and/or an interventional cardiac procedure.

In various embodiments, one or more operations executed on the one or more processors may be performed using a deep learning network or architecture or module. For example, the recognition of the tool and/or the generation of the segmented outline may be performed using a deep learning network or architecture, such as UNet or ResNet. In related embodiments, fitting of the shape of the tool onto the pixel mask may be performed using a computer vision algorithm and/or a shape fitting algorithm. Also, in various embodiments, the one or more processors may comprise a central processing unit, a graphical processing unit, or an application specific integrated circuit (ASIC). In additional or alternative embodiments, other suitable logic resources may be used in place of the processors including, for example, various analogue circuits, controllers, and state machines.

In many embodiments, the system further comprises the tool which can include a shaft and a shaped tip coupled to the shaft. The tool can comprise a surgical probe such as those described herein. According to one or more embodiments, the tool tip can have a rounded geometry, such as a spherical geometry. Also, according to some embodiments, the tip has a pattern, texture, or contrast (e.g., contrasting color relative to tissue at the selected tissue site) configured to enhance recognition of the tool tip by a deep learning network, machine learning algorithm, and/or computer vision algorithm executed on the one or more processors. In particular embodiments, the pattern or texture of the tip may correspond to various crisscross patterns and/or various geometric patterns such as hexagonal, pentagonal, or octagonal. Use of patterns, such as geometric patterns, can allow for the entire shape of the tip to be recognized by the imaging device even when only a portion is visible to the imaging device by reconstruction of the obscured portions of the tip based on the patterned portion that can be viewed. The reconstruction can be performed by one or more of a deep learning network, machine learning algorithm, or computer vision algorithm executed on one or more processors that are part of the system or operatively coupled to the system. The algorithms or learning networks can be trained to recognize the patterns on the probe. Alternatively, the particular probe tip pattern can be inputted into one or more of the algorithms or networks. Alternatively, the probe may include optical indicia of the pattern which can be detected by the medical imaging device and then read by the algorithm and/or deep learning network. The optical indicia can also include information on the shape, dimensions and size of the probe including the probe tip.

According to some embodiments, the shaft has a conical or other shape configured to have enhanced recognition by a deep learning network, a machine learning algorithm, and/or a computer vision algorithm executed on the one or more processors that are part of the system or operatively coupled to the system. In these and related embodiments, the tool tip has a spherical, hemispherical, or annular geometry.

Embodiments of the invention can be particularly useful for improving the accuracy and precision of the placement of digital landmarks at selected anatomical/tissue site during a minimally invasive procedure, such as an arthroscopic procedure, allowing for improved accuracy in landmark location and tissue measurements used for various task performed during the surgery, such as placement of sutures, screws, grafts or other implants. Such improvement can be particularly useful when the surgeon does not have a continuous and/or a clear field of view of the anatomical landmark and/or intended site for suture, screw, graft placement, etc.

In some embodiments, the operations performed by the processors further comprise identifying and labeling one or more elements in the video stream using at least one trained computer algorithm. In some embodiments, the one or more elements comprise one or more of an anatomical structure, a surgical tool, an operational procedure or action, or a pathology. In some embodiments, the identifying and labeling the one or more elements in the video stream comprises using one or more software modules (herein modules). In some embodiments, the one or more modules may comprise modules for performing video stream decomposition, tool recognition, anatomy recognition, tool tracking, gesture recognition, landmark point registration, or anatomy and landmark tracking. In some embodiments, the system recommends one or more landmarks based at least partially on the identified elements.

In some embodiments, the operations performed by the processor further comprise: storing the one or more sets of coordinates of one or more selected landmarks; changing a view of the display to omit the overlaid landmark from being displayed; reverting the view to a previous display; identifying the one or more set of coordinates for the one or more landmarks; and re-overlaying the one or more landmarks. In some embodiments, the operator activates the changing and the reverting steps. In some embodiments, changing a view step is activated automatically based on a change in an identified anatomical structure or pathology.

In some embodiments, the one or more sets of coordinates of the one or more landmarks are provided by an operator (e.g., a surgeon, interventional cardiologist, radiologists, etc.) intraoperatively, for example, by a foot pedal, or a button on the proximal end of the arthroscope or other medical imaging device. In some embodiments, the one or more sets of coordinates of the one or more landmarks are provided by an operator preoperatively. In some embodiments, the one or more sets of coordinates of the one or more landmarks are generated from one or more medical images of a subject. In some embodiments, the one or more medical images are radiological images of the subject. In some embodiments, the radiological images are from a joint or other boney structure of the subject. In some embodiments, the radiological images are associated with a shoulder, a knee, a hip, ankle, or elbow of the subject. In some embodiments, the radiological images are generated using fluoroscopy, magnetic resonance imaging (MRI), computed tomography (CT) scanning, positron emission tomography (PET) scanning, or ultrasound imaging.

In some embodiments, the video stream is provided by an arthroscope (or other imaging device) during the arthroscopic procedure. In various embodiments, the arthroscopic procedure may correspond to one or more of the following types of procedures (for which embodiments of the systems and modules may be configured for assisting with): ACL repair in a knee surgery; graft placement procedure, e.g., that is used in a superior capsule reconstruction of atorn rotator cuff, a decompression procedure; a removal of or a resection of one or more inflamed tissues; removal of or a resection of one or more frayed tendons where the video stream is monocular. In some embodiments, the video stream may be stereoscopic or monocular. Also, in various implementations, embodiments of the systems of the invention can be configured to toggle (or switch back and forth) between monocular or stereoscopic inputted video stream and associated outputted video overlays.

In some embodiments, the one or more computer processors receive the video stream from one or more camera control units using a wired media connection. In some embodiments, a latency between receiving the input from the digital camera and overlay the output and the videos stream is at most 40 milliseconds (ms) to accommodate a digital camera with about 24 frames per second (fps). In some embodiments, the latency between receiving the input from the digital camera and overlay the output and the videos stream is no more than a time between two consecutive frames from the digital camera.

In various embodiments, the one or more computer processors receive the video stream from one or more camera control units using a network connection which may be wired or wireless. In some embodiments, the interventional imaging device is a digital camera specialized for arthroscopic use. In some embodiments, the digital camera is mounted on a rigid scope, suitable for work in the arthroscopic joints. In some embodiments, the camera control unit is configured to control a light source and capture digital information produced by the digital camera. In some embodiments, the camera control unit converts the digital information produced by the digital camera into the video stream. In some embodiments, the camera control unit record the digital information produced by the digital camera in a memory device. In some embodiments, the memory device is a local memory device. In some embodiments, the memory device is a cloud-based memory device. In some embodiments, the digital camera is connected to a camera control unit which may be configured to overlay the output from with the one or more computer processors with the video stream.

In some embodiments, the system further comprises a display monitor which may be operatively coupled to the one or more computer or other processors. In some embodiments, the one or more computer processors comprise a central processing unit or a Graphical Processing Unit (also referred to as a GPU). In some embodiments, the system further comprises a mechanism to receive an input from at least one operator (to activate or stop marking the landmark) intraoperatively. In various embodiments, the mechanism is configured to receive the input via one or more of a push-button, a touchscreen device, a pointing device, (e.g., a mouse or a head mounted pointing device), a foot pedal, a gesture recognition system, or a voice recognition system. In some embodiments, the one or more landmarks are tracked during the arthroscopic or other medical procedure. In some embodiments, the tracking of one or more landmarks is associated with the set of coordinates of the one or more landmarks relative to an anatomical structure, an injury or pathology of the structure, an implant placed in the structure, and/or a repair of the structure.

In some embodiments, the display of the one or more landmarks are overlaid on the displaying video stream. In some embodiments, the one or more landmarks displayed as the relative anatomical structure is identified in the video stream. In some embodiments, the operator can select one or more landmarks to render the selected landmarks invisible temporarily or throughout the arthroscopic or other medical procedure.

In another embodiment, provided herein is a system for performing tissue landmarking during a minimally invasive medical procedure with the aid of a surgical tool, the system comprising: one or more processors, a memory to store instructions operable on the processors, an interface to receive image data from an imaging device positioned to image a scene including the tool when positioned at selected tissue location of a patient. The one or more processors can be configured to execute instructions stored in the memory to process image data received from the interface and to perform operations including tracking a position of a tip portion of the tool during the medical procedure. In some embodiments, the tip portion has a rounded geometry of a known dimension. Then, while tracking the tool, the one or more processors can be configured to perform at least one operation to recognize the tip portion separate from the remainder of the tool, where the recognition is based on the rounded geometry and the known dimensions, and adjust a rendering of the scene based on the recognition of the tip portion. The at least one operation may correspond to: generating a segments outline of the tip, for example, using a deep learning network and/or fitting a refined or smoothed outline onto the segmented outline using, for example, one or more of computer vision and/or shape fitting algorithm. The geometry of the tip may correspond to one or more of a sphere, hemisphere, or ring (e.g., an annular shape with a central opening). The tip may also include a pattern, texture, or contrast (e.g., contrasting color relative to tissue at the selected tissue site) configured to enhance recognition of the tool tip by a deep learning network, machine learning algorithm, and/or computer vision algorithm executed on the one or more processors.

Further provided herein are systems for assisting an arthroscopic or other surgical or medical procedure by allowing computer-implemented arbitrary landmark placement using radiological imaging, the system comprising one or more computer processors and one or more non-transitory computer-readable storage media storing instructions that are operable, when executed by the one or more computer processors, to cause the one or more computer processors to perform operations. In some embodiments, the operations comprise: receiving at least one radiological image of a subject; identifying one or more anatomical features in the at least one radiological image with a trained machine learning algorithm; generating a 3D representation of the identified anatomical features; receiving a location of one or more landmarks from an operator which may be done with the assistance of a probe or tool; overlaying the one or more landmarks on the 3D representation of the anatomical structures; and displaying the overlay on a displaying device to be used by the operator. In some embodiments, a probe may be positioned in the general area of the landmark and may be used to facilitate location of the landmark. The probe may have spherical or shaped tip and/or a patterned tip described herein so as to minimize error or inaccuracy in viewing of the probe (or other surgical tool) and tip such as that caused by differing viewing angles by the arthroscopic video camera and/or poor or suboptimal lighting of the probe tip. As used herein, suboptimal lighting generally refers to lighting inadequate or insufficient for the shape and/or outline of the probe (or other surgical tool tip) to be detected by embodiments of tool recognition algorithms described herein. Depending on the imaging modality, the probe can be constructed of materials that are opaque or reflective of the imaging modality, as well as other materials compatible with the imaging modality. For example, for ultrasound imaging, the probe can be constructed of echogenic materials. For radiological imaging, such as fluoroscopy, CAT scans, or PET scans, the probe can be made from radio-opaque materials. For MRI imaging, the probe can be constructed from MRI-compatible materials (e.g., non-ferrous materials). For non-video imaging, the tool or probe can be configured to be positioned outside of tissue (e.g., on the skin). In some embodiments, the probe can be configured to be positioned within tissue. Again, application of embodiments of the system described herein to the assistance of other medical procedures, including minimally invasive procedures such as endoscopic, laparoscopic, and interventional cardiovascular procedures, is contemplated.

In some embodiments, the anatomical features comprise a bony structure, such as shoulder joint, elbow, or knee, or soft tissue, such as a tendon or ligament. In some embodiments, the at least one radiological image comprises one or more of an MRI scan, a CT scan, a PET scan, an ultrasound image, or a combination thereof. In some embodiments, the at least one radiological image includes an image of a landmark which may include an image of a probe or tool positioned near or adjacent to the landmark. In some embodiments, the operations further comprise identifying the location of the landmark which may be done with assistance of the probe as described herein. In some embodiments, the operations further comprise recommending a location for a landmark based at least in part on the identified location of the landmark in at least one radiological image again which can be performed with aid of the probe to provide a point of reference, fiducial, or calibrating element or tool for dimensions of tissue at or near the intended landmark.

In some embodiments, the at least one radiological image or the one or more landmarks are blended with a video stream from an imaging device. In some embodiments, the blended image is displayed on a displaying device. In some embodiments, the displaying of the blended image occurs during the arthroscopic or other medical procedure. In some embodiments, the imaging device is an interventional imaging device, such as an ultrasound imaging device or a fluoroscopic imaging device. In various embodiments, the video stream may be monocular or stereoscopic, and the system can be configured recognize either to toggle back and forth between either type and generate the associated output accordingly.

Further provided herein are computer-implemented methods for assisting an arthroscopic or other minimally invasive medical procedure by the placement of one or more virtual (e.g., digital) landmarks at selected anatomical or other tissue locations. In various embodiments, the methods comprise: receiving a video stream from an imaging device; receiving one or more sets of coordinates of one or more landmarks; overlaying the one or more landmarks on the video stream; and displaying the overlay on one or more display devices intraoperatively to be used by an operator during the arthroscopic or other medical procedure. Application of embodiments of the above methods to the assistance of other minimally invasive procedures such as endoscopic, laparoscopic, and interventional cardiovascular procedures is also contemplated. In some applications, embodiments of the above methods and systems can be adapted for the placement of digital landmarks on cardiac and/or cardiovascular tissue (e.g., the atria, and the pulmonary vein) for performing radiofrequency (RF) or other ablative therapy to treat atrial fibrillation or other cardiac rhythm disorder. In particular embodiments, the systems and methods described herein can be configured to maintain the digital landmark(s) in place during beating of the heart so as compensate for wall motion or other movement of the atria, ventricles, and/or motion or movement of the pulmonary vein during the cardiac cycle. In additional or alternative embodiment of the above, methods for placement of virtual landmarks (including those using specialized probes described below) can be adapted and/or applied to a variety of open surgical procedure including orthopedic, cardiovascular, gastro-intestinal, neurological, and various oncology at any number of tissue site. In applications for oncology surgery, embodiments described herein can be configured to place landmarks corresponding to health tissue margins around a tumor so that tissue can be removed up to the healthy tissue margin.

In some embodiments, methods for placement of virtual landmarks at a selected location during a minimally invasive or other surgery involve the use of a specialized probe or other surgical tool. An embodiment of a method for performing tissue landmarking during a minimally invasive medical procedure, with the aid of a surgical tool having a tip of a selected/known geometry and size, comprises receiving a video stream from a medical imaging device, where the video stream encodes image data. The image data can be analyzed and the tool can be recognized from the image data. Then, a segmented tool outline, depicting the pixels where the tool is detected, can be generated and represented as a pixel mask. In some embodiments, just the pixels where the tool is detected are depicted; however, greater or fewer pixels can also be depicted. Then, a refined, fitted, and/or smoothed shape of the tool can be fitted onto the pixel mask where the refined, smooth, or fitted shape of the tool can be generated utilizing the selected geometry and size of the tool tip (e.g., diameter or other dimension of the tool), where the fitted shape minimizes error or inaccuracy in a shape of the pixel mask. Then, an intended location of the landmark is mapped onto tissue where the tissue mapping is based on the geometry and size of the tool tip. Then, a digital landmark is generated at a selected tissue location where the tool tip is positioned where the digital landmark is generated and positioned at the selected location utilizing the fitted tool shape and mapped location to generate a digital Then, the digital landmark is overlaid onto the video stream where the landmark can be displayed. The landmarks overlay can be performed in real-time with the performance of the medical procedure. In some embodiments, the landmarks overlay can be delayed by a selected amount of time (e.g., 0.5 to 2 seconds) or can be placed postoperatively so that the procedure video can be reviewed for analysis and/or medical education purposes. In particular embodiments, the landmarks can be placed postoperatively so that the procedure video can be compared to subsequent exploratory surgery videos of the anatomical structure operated on. In some embodiments, one or more of the above operations can be performed using software modules implemented by one or more processors such as micro-processors and/or graphical processing units. In alternative embodiments, one or more of the above operations can be implemented using other logic resources including various analogue based logic devices including, for example, various analogue controllers, states devices and the like.

In some embodiments, the method further comprises identifying and labeling one or more elements in the video stream using at least one trained computer algorithm, where the one or more elements comprise an anatomical structure, a surgical tool, an operational procedure or action, or a pathology. In some embodiments, identifying and labeling the one or more elements in the video stream comprises using one or more modules which may correspond to software modules stored in memory and operable on one or more processors. In some embodiments, the one or more modules comprise one or more modules for video stream decomposition, tool recognition, anatomy recognition, tool tracking, gesture recognition, landmark point registration, or anatomy and landmark tracking. In some embodiments, the one or more landmarks are recommended based at least partially on the identified elements.

In some embodiments, the method further comprises: storing the one or more sets of coordinates of one or more landmarks; changing a view of the display to omit the overlaid landmark from being displayed; reverting the view to a previous display; identifying the one or more set of coordinates for the one or more landmarks; and re-overlaying the one or more landmarks. In some embodiments, the operator activates the changing and the reverting steps. In some embodiments, the changing a view step is activated automatically based on a change in an identified anatomical structure or pathology.

In various embodiments, the one or more sets of coordinates of the one or more landmarks is provided by an operator intraoperatively or preoperatively. In the former case, the location for the coordinates for the landmark may be facilitated by a tool or probe described herein. The tool or probe may include a tip with geometry, such as spherical geometry having a determined target point for the landmark. The determined target point can reduce error or inaccuracy in the determination of the tip geometry, the target point, the location of the landmarks due to, for example, variations in the viewing angle of the arthroscope (or other imaging device) relative to the tool and/or the tool tip. In various embodiments, the one or more sets of coordinates of the one or more landmarks is generated from one or more medical images of a subject, which, in various embodiments, can make use of a tool or probe for identification of the landmarks. In some embodiments, the one or more medical images are radiological images. In some embodiments, the radiological images are generated using fluoroscopy, MRI, or CT scanning. In some embodiments, the video stream is provided by an arthroscope during an arthroscopic procedure. In some embodiments, the arthroscopic procedure is used in a rotator cuff implant surgery. In some embodiments, the arthroscopic procedure is used in an ACL tunnel placement in a knee surgery. In some embodiments, the video stream is monocular. In some embodiments, the video stream is stereoscopic.

In various embodiments, the receiving of the one or more video streams from the digital camera is performed using a wired media connection. In some embodiments, a latency between receiving the input from the digital camera and displaying an overlay of the output and the videos stream is at most 40 milliseconds (ms), for example, to accommodate a digital camera with about 24 frames per second (fps). In some embodiments, the latency between receiving the input from the digital camera and overlaying the output and the video stream is no more than a time between two consecutive frames from the digital camera. In some embodiments, the receiving of the one or more video streams from the digital camera is performed using a network connection.

In various embodiments, the interventional imaging device is a digital camera which may be mounted on a scope device such as arthroscope, endoscope, laparoscope, cardioscope, and the like. In some embodiments, the digital camera or other imaging device is operatively coupled to a camera control unit configured to control a light source and capture digital information produced by the digital camera. In some embodiments, the camera control unit is configured to convert the digital information produced by the digital camera into the video stream. Also in some embodiments, the camera control unit is configured to record the digital information produced by the digital camera in a memory device which may be a local memory device resident or operatively coupled to a computer system that performs one on more operations/steps of methods described herein or remote memory device, such as a cloud-based memory device. In some embodiments, the digital camera is connected to a camera control unit. In some embodiments, the video stream is received from the camera control unit by the one or more computer processing units to be processed. In some embodiments, the camera control unit is configured to overlay the output from the process by one or more computer processing units onto the video stream. In some embodiments, systems described herein further comprises a display monitor.

In some embodiments, the method comprises utilizing a mechanism to receive an input from an operator to activate or stop marking the landmark intraoperatively. In some embodiments, the mechanism may be configured to receive the input via a push-button, a touchscreen device, a foot pedal, a gesture recognition method, or a voice recognition method.

In some embodiments, the one or more landmarks are tracked during the arthroscopic or other medical procedure (e.g., an endoscopic, laparoscopic, cardioscopic procedure). In some embodiments, the tracking of one or more landmarks is associated with the set of coordinates of the one or more landmarks relative to an anatomical structure, an injury or pathology of an anatomical structure, an implant in an anatomical structure, or a repair of an anatomical structure. In some embodiments, the displaying of the one or more landmarks is blended with the displaying the video stream. In some embodiments, the one or more landmarks are displayed as the relative anatomical structure is identified in the video streaming. In some embodiments, the operator can select to render the one or more landmarks invisible temporarily or throughout the arthroscopic procedure.

In another aspect, provided herein are computer-implemented methods for assisting an arthroscopic or other medical procedure by arbitrary landmark placement using radiological imaging. In some embodiments, the methods comprise: receiving a radiological image of a subject; identifying one or more anatomical features in the radiological image using a trained machine learning algorithm; generating a 3D representation of the identified one or more anatomical features; receiving a location of one or more landmarks from an operator; overlaying the one or more landmarks on the 3D representation of one or more anatomical features; and displaying the overlay on a displaying device to be used by the operator. Application of embodiments of the methods to the assistance of other medical procedures including minimally invasive procedures, such as endoscopic, laparoscopic, and interventional cardiovascular procedures is also contemplated.

In some embodiments, the anatomical features comprise a bony structure or a tendon. In some embodiments, the radiological image comprises one or more of an MRI scan, a CT scan, or a combination thereof. In some embodiments, the radiological image includes an image of a landmark. In some embodiments, the method further comprises identifying the location of the landmark.

In some embodiments, the method further comprises recommending a location for a landmark based at least in part on the identified location of the landmark in the radiological image. In some embodiments, the radiological image or the one or more landmarks are overlaid on the video stream from an imaging device. In some embodiments, the blended image is displayed on a displaying device. In some embodiments, the displaying of the blended image is during an arthroscopic procedure. In some embodiments, the imaging device is an interventional imaging device. In some embodiments, the video stream is monocular. In some embodiments, the video stream is stereoscopic.

Further provided herein is a non-transitory computer readable medium comprising machine executable code that, upon execution by one or more computer processors, implements any of the methods above or elsewhere herein.

Further provided herein is a system comprising one or more computer processors and computer memory coupled thereto. The computer memory comprises machine executable code that, upon execution by the one or more computer processors, implements any of the methods above or elsewhere herein.

Additional aspects and advantages of the systems, devices, and methods described herein will become readily apparent to those skilled in this art from the following detailed description, wherein only illustrative embodiments are shown and described. As will be realized, the systems, devices, and methods described herein are capable of other and different embodiments, and its several details are capable of modifications in various obvious respects, all without departing from the systems, devices, and methods described herein. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The novel features of the present invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows a schematic example of a hardware configuration of a system for assisting an arthroscopic procedure by allowing computer-implemented arbitrary landmark placement, according to some embodiments.

FIGS. 2A-2B show examples of landmark placement on a model femoral condyle, according to some embodiments.

FIG. 3 shows a schematic of an example flow chart of a landmark placement system, according to some embodiments.

FIG. 4 shows a schematic of an example workflow of landmark placement using a preoperative image, according to some embodiments.

FIG. 5 shows a schematic of an example workflow of a system to recommend a landmark placement, according to some embodiments.

FIG. 6 shows a schematic flowchart of an example system to process a stereoscopic video stream, according to some embodiments.

FIG. 7 shows a computer system that is programmed or otherwise configured to implement methods provided herein, according to some embodiments.

FIG. 8 shows an example of placing a landmark and compensating for an occlusion, according to some embodiments.

FIG. 9 shows an example of a landmark being cleared from an object, according to some embodiments.

FIGS. 10A-10B show an example of stabilizing a landmark against a movement of a camera with respect to an anatomical structure, according to some embodiments.

FIGS. 11A-11B show an example of feature detection, according to some embodiments.

FIGS. 12A-12B are perspective views illustrating the impact of tool angle on embodiments of algorithms for recognition of surgical tools used in the operating field.

FIG. 13 is a perspective view illustrating an embodiment of a probe or other surgical tool having spherical tip which may be used in embodiments described herein for facilitating location/recognition of the tool tip and location, and placement of a digital landmark or fiducial.

FIG. 14 is a flow chart illustrating an embodiment of a method for recognizing a probe or other surgical tool and creating outlines of the tool using deep learning networks and computer vision and/or shape fitting algorithms.

FIG. 15 is a flow chart/block diagram illustrating embodiments of training and operational modes described herein used to training various embodiments of algorithms and/or learning networks used to identify and map the position of embodiments of the surgical probes and tools used for facilitating the placement of digital landmarks at a target anatomical/tissue site.

FIGS. 16A-16C are side views illustrating embodiments of surgical tools having a conical shaft and hemispherical (FIG. 16A), spherical (FIG. 16B) and ring-shaped (annular) tips (FIG. 16C) for facilitating location/recognition of the tool tip and location and placement of a digital landmark or fiducial.

FIGS. 17A-17C are side views illustrating embodiments of a surgical tool having have a spherical tip and various patterns (FIGS. 17A-17B) and/or textures FIG. 17C for facilitation location/recognition of the tool and/or tool tip and location and placement of a digital landmark or fiducial.

### DETAILED DESCRIPTION

While various embodiments of the present invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

Various embodiments provided are computer-implemented medical systems, devices, and methods for assisting surgeons in an intraoperative setting using AI. The systems, devices, and methods disclosed herein may improve upon existing methods of surgical landmark placement by providing a fast and reliable classification (e.g., in real-time) of various elements involved in a surgical operation (e.g., surgical tools, anatomical features, operation procedures) and placement of a virtual landmark (e.g., a digital landmark in the form of a colored dot or other marking) with high precision and accuracy based on the classification of various elements. For example, systems, devices, and methods provided herein may use AI methods (e.g., machine learning, deep learning) to build a classifier which improves a real-time classification of elements involved in a surgical operation and identifies a location of a landmark by intraoperative command from an operator (e.g., using a surgical probe) or by processing preoperative medical images (e.g., on MRI, CT scan, or fluoroscopy), where the preoperative medical images contain a landmark. An AI approach may leverage large datasets in order gain new insights from the datasets. The classifier model may improve real-time characterization of various elements involved in an operation which may lead to higher operation success rate. The classifier model may provide an operator (e.g., surgeon, operating room nurse, surgical technician) with information for more accurate placement of a virtual landmark which eliminates the shortcomings of a physical landmark. According to various embodiments, the virtual landmark (also referred to as a digital landmark) can be trackable, removeable, or otherwise changeable (in shape, color size, etc.), which may be done on demand. In addition to these benefits and/or advantages, the virtual landmark does not inhibit physical movement of the surgical tools during the surgery and is not necessarily obscured by surgical tools or other objects placed in the surgical field of view. According to various embodiments of the systems and methods described herein, the virtual landmark can be overlaid onto a video stream or other imagery of the surgery, which may be done on demand (e.g., to be displayed or not display) based on an input from an operator (e.g., by a foot pedal, switch, or a voice command).

In some embodiments, placement of the virtual landmark can be performed or facilitated with the use of a specialized probe or related surgical tool having a tip with a selected geometry and shape. In various embodiments, the geometry can have a spherical, rounded, or other shape selected such that the view and recognition of the tool tip is not affected by the viewing angle of the tool tip by a surgical imaging device, such as an arthroscope, endoscope, laparoscope, and the like. In use, embodiments described herein utilizing specialized probes result in improved accuracy and precision in placement of the digital landmark, and in turn, in measurements (e.g., of an anatomical dimension) and other operations (determination of suture placement or implant size) based on the landmark. In particular, use of specialized probes described herein can reduce errors caused by: i) differing viewing angles of the probe tip and/or orientation of the probe which may distort the shape of the probe tip relative to the video camera's (or other imaging device) perspectives; and ii) poor or sub-optimal lighting which may not fully illuminate the entire probe tip and/or resulting in the tool recognition module or related algorithm not being able to detect the entire outline or shape of the probe tip. Also in various embodiments, machine learning algorithms can be used to determine a target point of the tool tip (i.e., the intended point where the digital landmark is to be placed) based on a geometry of the tool tip, e.g., in the case of a sphere, hemispherical, or ring-shaped tip, the target point would be the center of the sphere, hemisphere, or ring. In use, these and related embodiments can also improve the accuracy and precision of placement digital landmark because the target point is precalculated and not affected by viewing angle or other visual error or artifact.

Reference will now be made in detail to various embodiments, examples of which are illustrated in the accompanying drawings. In the following detailed description, numerous specific details are set forth in order to provide a thorough understanding of the present invention and the described embodiments. However, the embodiments of the present invention are optionally practiced without these specific details. In other instances, well-known methods, procedures, components, and circuits have not been described in detail so as not to unnecessarily obscure aspects of the embodiments. In the drawings, like reference numbers designate like or similar steps or components.

The terminology used herein is for the purpose of describing particular embodiments only and is not intended to be limiting of the claims. As used in the description of the embodiments and the appended claims, the singular forms “a”, “an”, and “the” are intended to include the plural forms as well, unless the context clearly indicates otherwise. It will also be understood that the term “and/or” as used herein refers to and encompasses any and all possible combinations of one or more of the associated listed items.

As used herein, the term “if” is optionally construed to mean “when” or “upon” or “in response to determining” or “in accordance with a determination” or “in response to detecting,” that a stated condition precedent is true, depending on the context. Similarly, the phrase “if it is determined [that a stated condition precedent is true]” or “if [a stated condition precedent is true]” or “when [a stated condition precedent is true]” is optionally construed to mean “upon determining” or “in response to determining” or “in accordance with a determination” or “upon detecting” or “in response to detecting” that the stated condition precedent is true, depending on the context.

As used herein, and unless otherwise specified, the term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, 0.1%, or 0.05% of a given value or range.

As used herein, the terms “comprises,” “comprising,” or any other variation thereof, are intended to cover a nonexclusive inclusion, such that a process, method, article, or apparatus that comprises a list of elements does not include only those elements but may include other elements not expressly listed or inherent to such process, method, article, or apparatus.

As used herein, the terms “subject” and “patient” are used interchangeably. As used herein, the terms “subject” and “subjects” may refer to a human being. In certain embodiments, the subject is undergoing a surgical operation. In certain embodiments, the subject is 0 to 6 months old, 6 to 12 months old, 1 to 5 years old, 5 to 10 years old, 10 to 15 years old, 15 to 20 years old, 20 to 25 years old, 25 to 30 years old, 30 to 35 years old, 35 to 40 years old, 40 to 45 years old, 45 to 50 years old, 50 to 55 years old, 55 to 60 years old, 60 to 65 years old, 65 to 70 years old, 70 to 75 years old, 75 to 80 years old, 80 to 85 years old, 85 to 90 years old, 90 to 95 years old or 95 to 100.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The term “surgical AI” or “surgical AI module”, as used herein, generally refers to a system, device, or method that uses Artificial Intelligence algorithms to assist before, during, and/or after a surgical operation. A surgical AI module can be defined as a combination of input data, machine learning or deep learning algorithms, training datasets, or other datasets.

The term “machine learning”, as used herein, may generally refer to computer algorithms that can improve automatically over time. Any description herein of machine learning can be applied to Artificial Intelligence, and vice versa, or any combination thereof.

As used herein, the terms “continuous,” “continuously” or any other variation thereof, generally refer to a substantially uninterrupted process or a process with time delay that is acceptable in the context of the process.

The terms “video stream”, or “video feed”, as used herein, may refer to data generated by a digital camera. Video feed may be a sequence of static or moving pictures.

The terms “region,” “organ,” “tissue,” or “structure”, as used herein, may generally refer to anatomical features of the human body. A region may be larger than an organ and may comprise an organ. An organ may comprise one or more tissue types and structures. A Tissue may refer to a group of cells structurally joined to complete a common function. A structure can refer to a part of a tissue. In some embodiments, a structure may refer to one or more parts of one or more tissues joined together to create an anatomical feature.

The terms “surgical field of view,” or “field of view,” as used herein, may refer to the extent of visibility captured by an interventional imaging device. Field of view may refer to the extent of visual data captured by a digital camera that is observable by human eye.

The term “decision,” as described herein, may refer to outputs from a machine learning or AI algorithm. A decision may comprise labeling, classification, prediction, etc.

The term “interventional imaging device,” as used herein, generally refers to an imaging device used for medical purposes. The interventional imaging device may refer to an imaging device that is used in a surgical operation e.g., an arthroscope, a cardioscope, an endoscope, a laparoscope, or other like device. The surgical operation, in some embodiments, may be a simulation of an operation or other medical procedure.

The term “operator,” used herein, may refer to a medical professional involved in a surgical operation. An operator can be a surgeon, an operating room nurse, a surgical technician.

The term “landmark”, “arbitrary landmark”, “virtual landmark”, and “fiducial marker” are as used interchangeably herein to refer to marks used to guide surgical or other medical procedures.

In one aspect, provided herein is a system for assisting an arthroscopic or other minimally invasive procedure by allowing computer-implemented arbitrary landmark placement. The system may comprise one or more computer processors and one or more non-transitory computer-readable storage media storing instructions that are operable, when executed by the one or more computer processors, to cause the one or more computer processors to perform operations comprising: receiving a video stream from an arthroscopic or other interventional medical imaging device; receiving one or more sets of coordinates of one or more landmarks; overlaying the one or more landmarks on the video stream; and displaying the overlay on one or more display devices intraoperatively to be used by an operator during the arthroscopic procedure. In some embodiments, an operator (e.g., a surgeon) provides the one or more sets of coordinates of one or more landmarks preoperatively.

The video stream may be provided by the arthroscopic imaging device during the arthroscopic procedure. In some embodiments, the arthroscopic imaging device comprises a digital camera. The video stream may be obtained from a digital camera specialized for an arthroscopic procedure. The digital camera may be mounted on a rigid scope, suitable for work in the arthroscopic joints. The scope may comprise an optical fiber which illuminates a field of view of the surgery. The digital camera may be mounted to a camera control unit. In some embodiments, a camera control unit is configured to capture digital information produced by the digital camera. In some embodiments, the camera control unit converts the digital information produced by the digital camera into the video stream. In some embodiments, the camera control unit is configured to control a light source. In some embodiments, the camera control unit is configured to record the digital information produced by the digital camera in a memory device. In some embodiments, the memory device used to record the digital information is a local memory device. In some embodiments, the camera control unit is configured to overlay the output from the one or more computer processors with the video stream. In some embodiments, the memory device is a remote or cloud-based memory device. The camera control unit may send the video stream to the one or more computer processors. In some embodiments, the system comprises more than one camera control units. In some embodiments, the system comprises 2, 3, 4, 5, or more camera control units. The one or more camera control units may send the video streams to the computer processors via network connection or a wired media connection. The video stream may be stereoscopic or monocular. In some embodiments, the system further comprises a display monitor. In some embodiments, the system comprises a mechanism to receive an input from the at least one operator (e.g., to activate or stop marking the landmark) intraoperatively. In some embodiments, the mechanism receives the input via a push-button, a touchscreen device, a foot pedal, a gesture recognition system, or a voice recognition system.

In some embodiments, the one or more sets of coordinates are provided during the surgery using a digital pointer (e.g., a computer mouse or related device) that can mark an image in the video stream to select a point or a region for performing surgery and/or a surgical action (e.g., tissue resection, ablation, etc.). In some embodiments, an operator (e.g., a surgeon) provides the one or more sets of coordinates intraoperatively by indicating the desired location using a standard surgical probe. In some embodiments, after the coordinates of a desired location are selected or indicated, an operator can issue a command so that the system can register the location. In some embodiments, the system receives the register command from the operator via a push-button, a touchscreen device, a foot pedal, a gesture recognition system, or a voice recognition system.

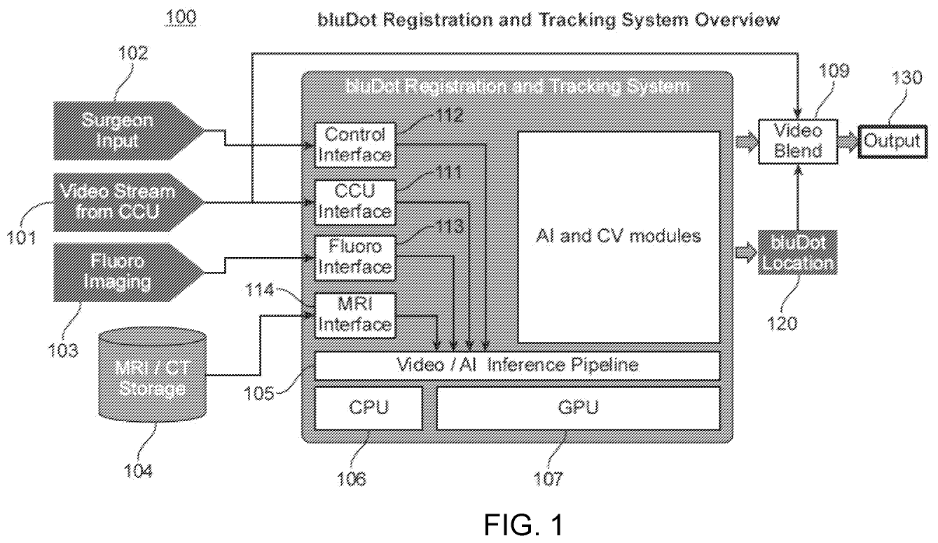

FIG. 1 shows a schematic example of a hardware configuration of the system described herein. The example system 100 may comprise a plurality of inputs. The plurality of inputs may comprise a video stream input 101, an operator (e.g., surgeon) input 102, and one or more preoperative imaging inputs. The preoperative imaging inputs may comprise a fluoroscopy imaging input 103, a medical data system (e.g., radiology imaging such as MRI, or CT scan) input 104. In some embodiments, each of the plurality of inputs is connected to a corresponding interface. For example, video stream input 101 may be connected to a camera control unit (CCU) 111, operator input 102 may be connected to a control interface 112, fluoroscopy imaging input 103 may be connected to a fluoroscopy interface 113, or medical data system input 104 may be connected to a medical data system (e.g., MRI or radiology imaging) interface 114. Each of the interfaces may be configured to receive an input from their corresponding inputs. The system 100 may support other external interfaces to receive input in various modalities from the surgeon, clinical data systems, surgical equipment, etc. The plurality of inputs received by a plurality of interfaces may then be sent to a processing unit to be processed using an artificial intelligence (AI) pipeline. In some embodiments, the processing unit may comprise a central processing unit (CPU) 106, a graphical processing unit (GPU) 107, or both. In some embodiments, a CPU or a GPU comprises a plurality of CPUs or GPUs. The CPU or GPU may be connected to the plurality of interfaces via a media connector (e.g., an HDMI cable, a DVI connector). The CPU or GPU may be connected to the plurality of interfaces (e.g., surgical video camera) over network connection (e.g., TCP/IP), which may provide more flexibility with less wired connections. In some embodiments, the latency in video processing and playback may be higher when the connection is via network as compared to a media connector. In some embodiments, the network connection may be a local network connection. The local network may be isolated including a set of predefined devices (e.g., devices being used in the surgery.) Lower latency may be more desirable for real-time feedback (e.g., during a surgery). In some embodiments, a system setup with higher latency can be used for training purposes (e.g., a mock surgery). The AI pipeline may comprise one or more machine learning modules or AI modules comprising one or more computer vision (CV) modules. In some embodiments, the AI and CV modules are supported by a video and AI inferencing pipeline (VAIP) 105. In some embodiments, VAIP 105 supports the AI and CV modules and manages the flow of control and information between the modules. VAIP 105 may comprise a configuration file comprising instructions for connecting and managing the flow. VAIP 105 may support execution of the AI algorithms on a GPU 107. VAIP 105 may also support direct media interfaces (e.g., HDMI, or DVI). One or more outputs of the plurality of inputs processed by the AI pipeline may be generated, comprising a landmark (bluDot) location 120 and one or more feature elements identified from the plurality of inputs to generate a video blend 109. The one or more outputs may be overlaid onto the video stream input 101 to generate an output 130. In some embodiments, the system 100 comprises a display monitor. In some embodiments, output 130 is displayed on a display monitor (e.g., a monitor, a television (TV)). In some embodiments, the system comprises a displaying device. In some embodiments, landmark location 120 and video blend 109 are sent back to the CCU to be overlaid onto the video stream to generate output 130.

In some embodiments, the arthroscope may generate consecutive images (e.g., a video feed) at a rate of at least about 10 frames per second (fps). In some embodiments, there is a latency in the system, which is the time of required to receive an image (e.g., a video feed) and provide an overlay (e.g., a processed image). In some other cases, two consecutive frames from the video stream (e.g., video stream input 301) may be generated at a speed of 1/fps (frames per second). In some embodiments, the latency in the system is at most 1/fps. The latency of the system may be less than the inverse of the rate of consecutive image generation of the surgical camera. For example, when the input signal is streaming at 20 frames per second, the latency may be equal or less than 1/20 (1/fps) or 50 ms. In some embodiments, the latency may comprise a period of rest in the system.

In some embodiments, the operations may further comprise identifying and labeling one or more elements in the video stream using a trained computer algorithm, where the one or more elements comprise one or more of an anatomical structure, a surgical tool, an operational procedure or action, or a pathology. In some embodiments, identifying and labeling the one or more elements in the video stream comprises using one or more AI modules. In some embodiments, the one or more AI module may comprise one or more modules for video stream decomposition, tool recognition, anatomy recognition, tool tracking, gesture recognition, landmark point registration, or anatomy and landmark tracking. In some embodiments, the system recommends one or more landmarks based at least partially on the identified elements. In some embodiments, the system recommends one or more landmarks based at least partially on the identified elements.

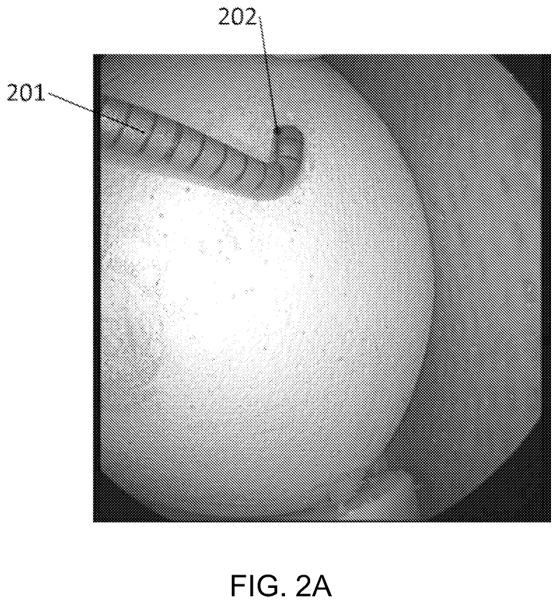

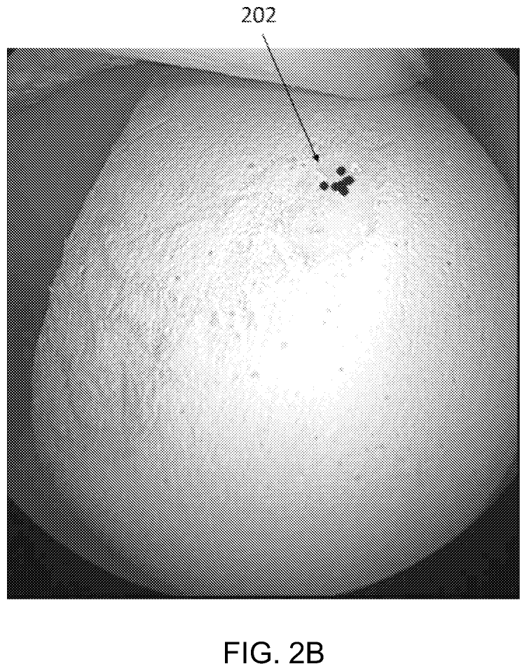

FIGS. 2A-2B show examples of landmark placement on a model femoral condyle. In some embodiments, as shown in FIG. 2A, an operator (e.g., a surgeon) indicates a desired location of a landmark using a standard surgical probe 201. The operator may then activate a register command to the system to register the desired location. The location of the landmark can be visualized on the screen displaying the video stream of the surgery with a dot 202 (e.g., a blue dot). In some embodiments, the system saves the location of the landmark and tracks the landmark throughout the surgery. The dot visualizing a landmark can be displayed on the screen or removed from the screen at any moment by the operator. In some embodiments, the landmark is one isolated dot. In some embodiments, landmark is a plurality of isolated dots 202, as shown in FIG. 2B. In some embodiments, the landmark is a virtual arbitrary pattern or a predefined shape. In some embodiments, the location of a landmark can be indicated and/or selected preoperatively. The landmark may be a location of an implant or an anchor location during arthroscopic procedures. In various embodiments, such arthroscopic procedure may correspond to one more or of a rotator cuff repair surgery; an ACL repair in a knee surgery; a graft placement procedure; a decompression procedure which may include removal or reshaping of bony structures to reduce pain; or a shoulder arthroscopic procedure involving placement of a graft such as those used in repair of a rotator cuff. In various embodiments, one or more of the shape, size, and color of the digital landmark can be customized for each of the aforementioned procedures to optimize the visibility and usability of the landmark e.g., for suture or anchor placement or for making measurements or other use described herein. For example, different colored landmarks can be used depending upon whether the tissue in the background is bone, cartilage, tendon, muscle or other tissue. Additionally, various embodiments of the specialized probes or other surgical tools used for facilitation placement of the digital landmark (described herein) can also be customized in shape, size, color and pattern or texture (including those qualities of the tool tip) for each surgical application along with algorithms for placement of the digital landmark.

In some embodiments, the arthroscopic procedure comprise removal, or resection of an inflamed tissue and/or frayed tendons. In some embodiments, the arthroscopic procedure is used in a removal of or a resection of one or more inflamed tissues. In some embodiments, the arthroscopic procedure is used in a removal of or a resection of one or more frayed tendons where the video stream is monocular. For example, radiological imaging or other imaging methods, such as fluoroscopic imaging, can be used to locate the place of a landmark. An image obtained from these imaging methods can be provided to the system to be overlaid on the video stream during the surgery. In some embodiments, the system shows a latent anatomical structure or pathology; for example, the operator (e.g., a surgeon) may protect the ureter as the system visualizes the location of the ureter although the ureter may not be exposed during a surgical procedure. For example, the system can ingest information from an external system, such as a fluoroscopic imaging system. The landmark may then take the form of the vascularity rendered visible by the fluorescent dye imaged using the fluoroscopic imaging system. The system may continue to retain and track vascularity during the procedure (e.g., arthroscopic surgery).

In some embodiments, the system 100 operates on stored video content. A video recording of an arthroscopic surgery can be played back and sent to an interface. The system may then overlay any landmark on the video stream as explained herein. In some embodiments, the landmark placement on a recording of a surgery is used for training purposes. In some embodiments, the system operates on stereoscopic video streams. In some embodiments, the system can be used during a robotic arthroscopic procedure (e.g., a surgery). In some embodiments, a view of the display may be changed. In some embodiments, by changing a view a landmark that is overlaid on a video stream can be omitted from being displayed. In some embodiments, the operator can select to render the landmark invisible temporarily or throughout the arthroscopic procedure. The operator may revert the view to a previous display. The operator may identify new sets of coordinates for a landmark. The new landmark may be overlaid on to the video stream to be displayed. A plurality of landmarks may be selected to be displayed simultaneously or one at a time. In some embodiments, a change in the view is automatic. In some embodiments, changing a view may be cause by the AI pipeline identifying an anatomical structure or pathology.

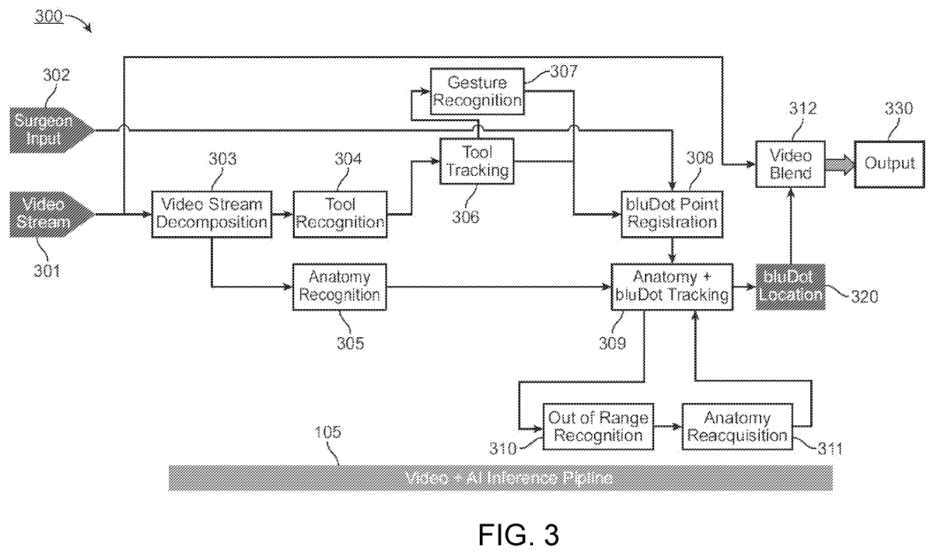

In some embodiments, a set of coordinates of the landmark is provided by an operator intraoperatively. FIG. 3 shows a schematic of an example flow chart of a landmark placement system 300. The system may comprise a plurality of modules which operate on the video stream input 301 generated by an arthroscopic camera and an input received from an operator 302 (e.g., a surgeon). In some embodiments, video stream input 301 is processed by a video stream decomposition module 303 comprising a CV algorithm to decompose a video stream into a series of images. The series of images may be stored in a memory device. One or more images from the series of images may be provided to one or more downstream component comprising a tool recognition module 304 or an anatomy recognition module 305. In some embodiments, video stream decomposition module 303 outputs an image of the field of view of the surgery.

In some embodiments, the tool recognition module 304 uses an AI network to recognize surgical tools in the field of view. Non-limiting examples of the AI network used in tool recognition module 304 may comprise Mask R-CNN, UNET, ResNET, YOLO, YOLO-2, or any combination thereof. In some embodiments, the AI networks are trained to recognize surgical tools of interest using machine learning training comprising architecture-specific training techniques. In some embodiments, the trained AI network detects the presence of a surgical tool in an image and outputs a mask. The mask may be a set of pixels extracted from the input image, which indicate the precise outline of the surgical tool. In some embodiments, the AI network outputs a box (e.g., a rectangular region) in which the tool is detected or displayed.

In some embodiments, the anatomy recognition module 305 uses an AI network to recognize an anatomical structure in a field of view. Non-limiting examples of the AI network used in anatomy recognition module 305 may comprise Mask R-CNN, UNET, ResNET, YOLO, YOLO-2, or any combination thereof. In some embodiments, the AI networks are trained to recognize anatomical structures of interest using architecture-specific training techniques. In some embodiments, the trained AI network recognizes anatomical structures in the field of view. In some embodiments, the trained network outputs pixel masks, which may indicate the precise outline of the recognized anatomical structure. In some embodiments, the trained network outputs a box (e.g., a rectangular region) in which the tool is detected or is displayed.

In some embodiments, an output from tool recognition module 304 is provided to a tool tracking module 306. In some embodiments, the tool tracking module 306 tracks the motion of the one or more tools identified by the tool recognition module 304. In some embodiments, a position of a tool (e.g., an instantaneous position of the tool) may be stored in a memory (e.g., a buffer). In some embodiments, tool tracking module 306 uses CV algorithms to compute the velocity and acceleration of the tool and stores these values in the memory. This data may be stored as a fixed length array. In some embodiments, this array is stored in the time order that the values were captured. In some embodiments, the array is stored in descending order of time. The array may have a fixed length and with adding new data, an older entry may be dropped out of the array and the memory buffer. In some embodiments, adding a new entry causes the oldest entry to be dropped out of the array. An output of the tool tracking module 306 may comprise the mask of the recognized tool along with the array of the tool's velocity and/or acceleration. The tool tracking module 306 may supply a position or an array of the positions of one or more tool to a gesture recognition module 307 and a landmark registration module 308 (e.g., a bluDot point registration module).

In some embodiments, the gesture recognition module 307 uses an AI network comprising a memory (e.g., a recurrent neural network (RNN)), to interpret the movement of the tools. In some embodiments, the AI network is trained to recognize specific tools and/or identify specific movement patterns. For example, a tap can involve the tool moving in a specific manner relative to the background anatomy. In some embodiments, the surgeon can indicate a position of an arbitrary landmark by using a predetermined gesture using a surgical tool. Non-limiting examples of a gesture may comprise tapping, double tapping, triple tapping, wagging (e.g., moving a tool from left to right). In some embodiments, gesture recognition module 307 outputs a label of the gesture made by an operator using a tool. In some embodiments, gesture recognition module 307 recognizes a gesture made by the operator and generates a label of the name of the recognized gesture to be supplied to a downstream component, which may be landmark registration module 308.

In some embodiments, the landmark registration module 308 receives one or more inputs from tool tracking module 306 and/or gesture recognition module 307, as described herein. In some embodiments, the input from gesture recognition module 307 instructs landmark registration module 308 that a gesture from an operator is recognized. The gesture may then be mapped to an action. In some embodiments, the mapping is configured preoperatively and is loaded from a database when the system is initialized. Non-limiting examples of an action mapped by landmark registration module 308 may comprise to initiate, to replace, or to clear all. In some embodiments, landmark registration module 308 may be initiated to assign a unique identifier to a landmark (e.g., a dot, such as a blue dot, herein bluDot). An action comprising a command to clear one or more landmarks or clear all may activate landmark registration module 308 to update a list of one or more landmarks. An initiate action may trigger landmark registration module 308 to supply the location of the tool to anatomy and landmark tracking component 309 (e.g. anatomy+bluDot tracking). A replace action may trigger landmark registration module 308 to replace the data associated with the location of one or more landmarks with a location of a new landmark. A clear all action may trigger landmark registration module 308 to clear any landmark that is being displayed or is stored in the memory. In some embodiments, landmark registration module 308 receives a direct input from the operator to place, replace, or clear a landmark. The direct input may be provided using a digital or mechanical button, for example, a foot-pedal-press or the press of a dedicated button or switch on the arthroscopic device. In some embodiments, the CCU communicates the direct input through a custom interface to the VAIP 105 described herein. For example, a custom interface may comprise a gesture mapped to an action that is customized for an operator.

In some embodiments, landmark registration module 308 makes a distinction between the manner in which the landmarks are specified (e.g., preoperatively, intraoperatively via a gesture, intraoperatively via direct command from an operator, intraoperatively through the use of a specialized probe described herein) for rendering and/or recall purposes. For example, a landmark obtained from preoperative planning can be saved from deletion. In some embodiments, landmark registration module 308 supplies a set of coordinates of a tool identified by tool recognition module 304 in the image of the surgical field of view. The updated list in landmark registration module 308 may be passed down to a downstream anatomy and landmark tracking component 309, which may stop tracking and displaying landmarks that may be set for being cleared. For example, fluorescent dyes may be injected in the blood vessels to assist an operator (e.g., a surgeon) in identifying an artery or a vein. In some embodiments, the surgery is performed in close proximity to highly vascular regions, where nicking an artery or a vein can have serious consequences. An image of the identified arteries or veins (e.g., by using a dye) may be received and overlaid on the surgical video by VAIP as a landmark. A confidence may be calculated, substantially continuously, representing a certainty in the accuracy of VAIP to track and recall the landmark (e.g., identified arteries or veins). In some embodiments, the confidence may lower than a threshold, where the threshold may be about 80%, 70%, 60%, 50%, 40%, 30%, 20%, 10%, or lower. The threshold may be set by the operator. In some embodiments, the change in confidence is due to changes in the surgical field of view (e.g., operation being performed may change the anatomical structure). The system may then indicate that the confidence in tracking the landmark has diminished. The operator may feature the landmark by, for example, injecting dyes into the blood vessels. The system may then replace the previously identified landmark with newly identified landmark and overlay the landmark on the surgical image (e.g., video stream).

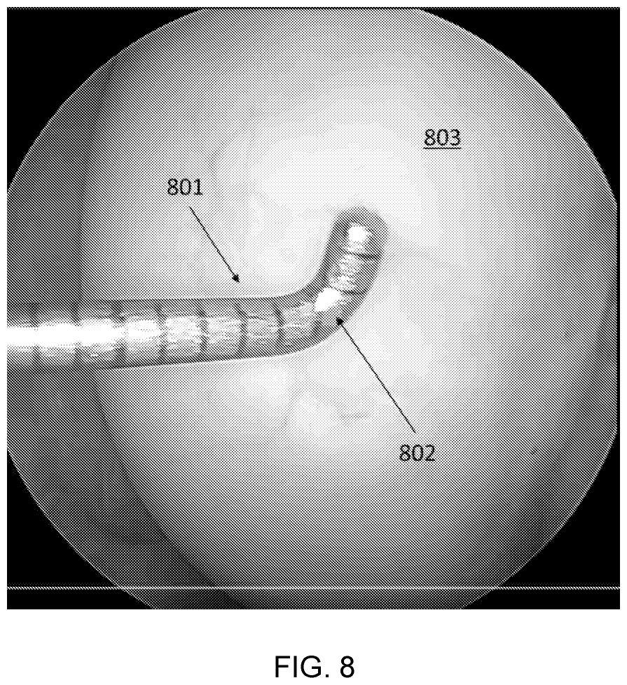

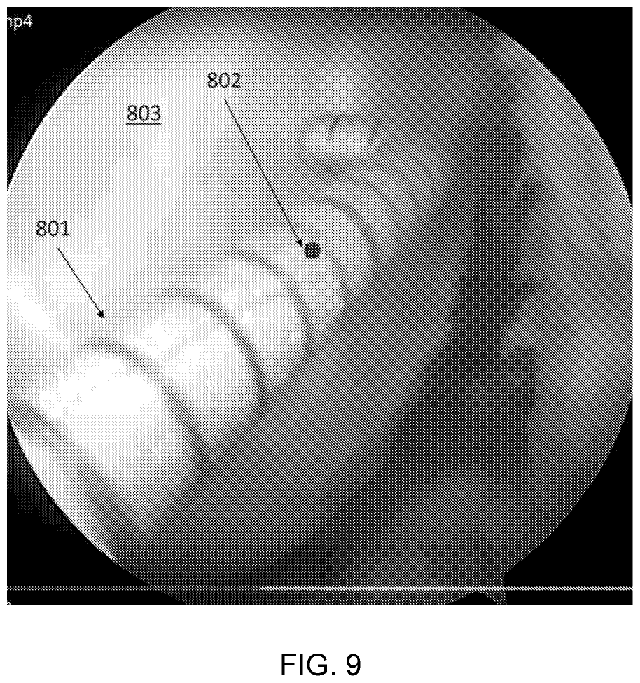

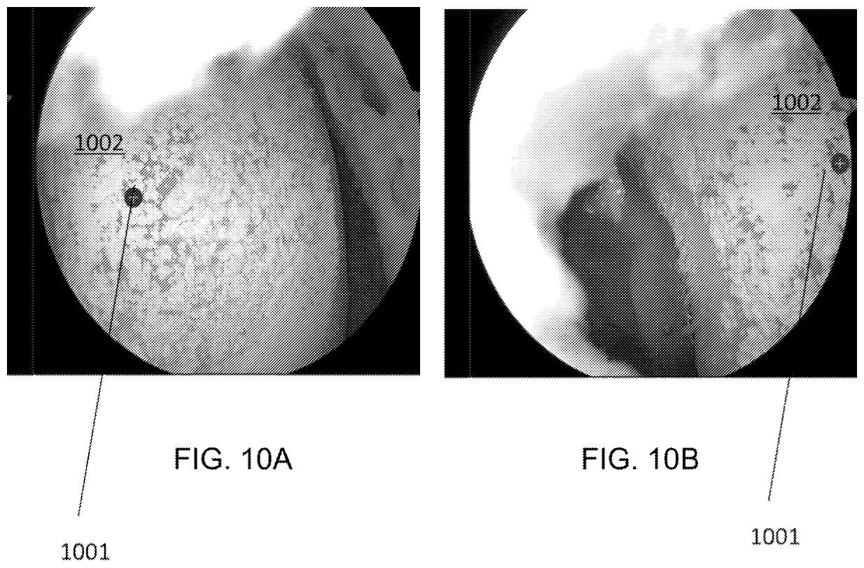

In some embodiments, the anatomy and landmark tracking component 309 receives an anatomy mask from anatomy recognition module 305 and/or an identified tool mask from tool recognition module 304 in a substantially continuous manner. In some embodiments, anatomy and landmark tracking component 309 receives an input from landmark registration module 308 that indicates an action, anatomy and landmark tracking component 309 performs a series of operations. The series of operations may comprise determining the superposition of the tool and the anatomical structure from the mask to determine over which anatomical structure the tool is placed or held. The coordinates of the tool and the anatomical structure may be used to identify the overlap of the tool with the anatomical structure. The series of operations may further comprise extracting a feature to locate a landmark (e.g., a bluDot) in relation to a location of one or more anatomical structures. In some embodiments, the feature comprises a detail on the image, which may comprise a pattern of vascularity, an edge of tissue, or a locally unique patch of pixels. Using the feature, the system may stabilize the landmark (e.g., bluDot) against a movement of the camera with respect to an anatomical structure (also shown in FIGS. 10A-10B). In some embodiments, the feature may comprise points on the tool in the surgical field of view, where the tool is moving independent of an anatomical structure. The feature on the tool may then be excluded in anatomy and landmark tracking component 309 to stabilize the landmark against movements of the tool (also shown in FIGS. 8 and 9).

In some embodiments, the landmark is initialized and designated at an initial position of the tool. In some embodiments, anatomy and landmark tracking component 309 identifies changes in a location of a feature on an anatomical structure and re-designates the location of the landmark. In some embodiments, anatomical structures are modeled as mildly deformable solids and suitable computational techniques are used to track the features on the anatomical structures. In some embodiments, anatomy and landmark tracking component 309 acquires a feature continuously and tracks a movement of the landmark on an expanded canvas. An expanded canvas may comprise the surgical field of views in different images acquired from the video stream that may be connected to one another to generate a larger field of view. In some embodiments, using the feature described herein, the system tracks the landmark with a high degree of certainty even if the landmark or the underlying anatomical structure move off the camera's field of view. In some embodiments, during surgery, the operator might move the camera away from the location of the landmark and surrounding tissues causing the operator to lose sight of the landmarks. In some embodiments, the location of the landmark needs to be re-acquire when the operator returns to the general area again. In some cases, an anatomical structure is recognized first, as described before, excluding any tools in the field of view. One or more features may be identified to recognize the location of the landmark according to an anatomical structure that has been recognized before.

For example, one or more feature points may be identified that may be separated by the anatomical structures on which the one or more feature points appear. When the surgeon re-enters a surgical field of view that has been analyzed in a previous image, the anatomical recognition module(s) may recognize the previously processed image. Upon matching the new coordinates of the feature points in the current image with the coordinates of the feature points in the previously processed image, the landmark may be placed in its location. The location of the landmark may be reestablished based on the feature points in the current image as well as the previously identified feature points. The feature points matching process may be repeated to increase the accuracy of landmark placement. This process may be performed using parallel computing (e.g., a GPU). The system may discard the feature points identified in the previously processed image and replace them with the feature points identified in the current image. The processed described herein may be performed using the anatomy and landmark tracking module 309, out of range recognition module 310, and/or anatomy reacquisition module 311.

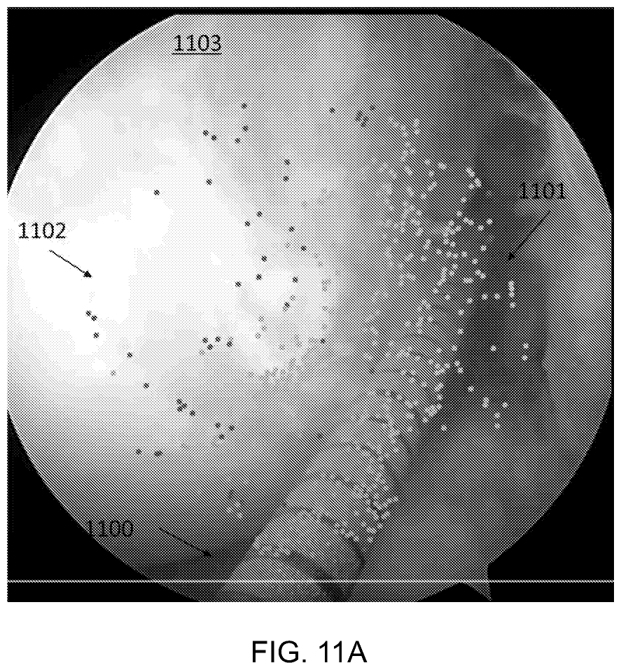

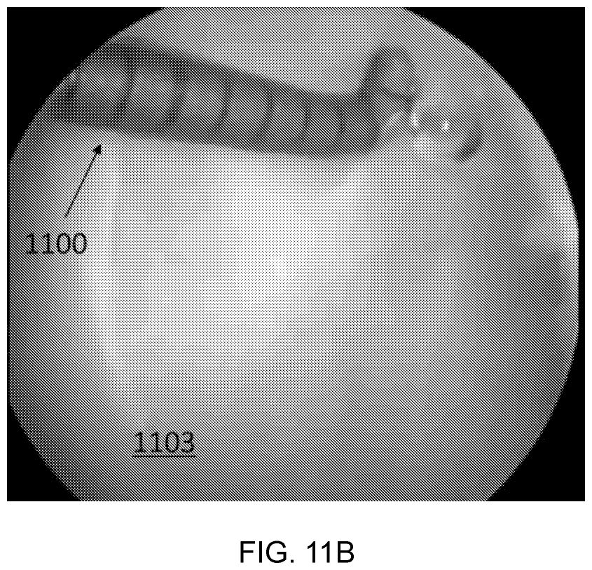

FIGS. 11A-11B shows an example of feature detection. For example, a plurality of features (or feature points) 1101 may be detected on a tool 1100 (shown as light gray points 1101 in FIGS. 11A-11B) may be distinguished from set of features 1102 recognized on the anatomical structure 1103 (shown as dark gray points 1102 in FIGS. 11A-11B). In some embodiments, during a surgical procedure, the surgical field of view may be altered. For example, a procedure may comprise debridement of soft tissue that may change the field of view. Once the tool is recognized and tracked (e.g., in real time), the feature points detected on the tool may be eliminated. In some embodiments, the feature points detected on the anatomical structure may be used to track the landmark. This tracking may improve or stabilize recognition of a landmark against tool movements that may block a landmark. FIG. 8 shows an example of an occlusion by the tool being ignored when overlaying landmark on a video or image. In some embodiments, bleeding or presence of body fluids may change the field of view. In some embodiments, the operations in anatomy and landmark tracking component 309 comprise continuously acquiring features from the field of view and discarding features that are missing in consecutive images to stabilize the landmark against the changes in the field of view from an action being performed in a procedure. The features may be acquired against an anatomical structure as reference. In some embodiments, anatomy and landmark tracking component 309 comprises an out of range recognition module 310 and an anatomy reacquisition module 311. In some embodiments, anatomy and landmark tracking component 309 updates the location of the landmark based on a feature in the observable portion of an anatomical structure. In some embodiments, as described herein, the field of view may be shifted excluding the anatomical structure or the landmark. As the camera pans back to the location of the landmark, anatomy and landmark tracking component 309 may increase the confidence of the position of the landmark by using out of range recognition 310 and anatomy reacquisition module 311, as described herein. The output of anatomy and landmark tracking component 309 comprises a location of the landmark (e.g., bluDot) in the field of view and/or within a frame or boundaries of an image being processed. The location of the landmark can be sent to landmark location module 320 (e.g., bluDot location). The landmark may then be overlaid on the output video stream 330. An example of the output video stream 330 is shown in FIG. 8. In some embodiments, the surgical field of view is about 3 centimeters (cm) to about 6 cm. In some embodiments, the surgical camera's (e.g., arthroscope) range of movement is about 3 cm to about 6 cm. In some embodiments, the range for stabilization is similar to the surgical camera's range of movement, which is about 3 cm to about 6 cm. In some embodiments, the precision in stabilizing the landmark against the changes in the field of view is about 1 millimeters (mm) to about 3 mm.

In some embodiments, the output from the landmark location module 320 is overlaid onto the video stream input from module 301 in a video blend module 312. The output from video blend module 312 may be displayed on output video stream 330 (e.g., with a screen, a monitor, a TV, a laptop screen, etc.). The output from 320 may be directed to the camera control unit to be scaled and overlaid onto the video stream of the procedure.

In another aspect, provided herein is a system for assisting an arthroscopic procedure by allowing computer-implemented arbitrary landmark placement using radiological imaging. The system may comprise one or more computer processors and one or more non-transitory computer-readable storage media storing instructions that are operable, when executed by the one or more computer processors, to cause the one or more computer processors to perform operations comprising: receiving a radiological image of a subject; generating a 3D representation of the radiological image; identifying an anatomical structure in the radiological image with a trained machine learning algorithm; receiving a location of a landmark from an operator; overlaying the landmark on the 3D representation of the radiological image; and displaying the overlay on a displaying device to be used by the operator.

In some embodiments, an operator identifies or set a location of a landmark during a preoperative surgery planning phase. In some embodiments, the landmark may be set by an operator (e.g., a surgeon) on a radiology images obtained from a subject. The landmark may then be supplied to a landmark registration module similar to landmark registration module 308 in FIG. 3. This may allow the operator to hide or display the landmark during the preoperative surgery planning phase.

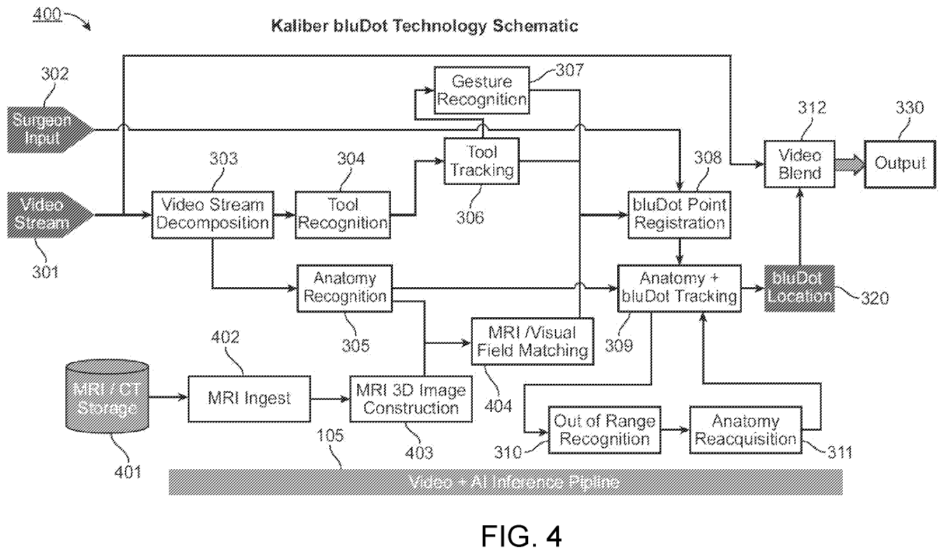

FIG. 4 shows a schematic of an exemplary workflow of landmark placement using a radiology imaging. As shown in FIG. 4, a plurality of modules may be added to the system 300 shown in FIG. 3 to allow using preoperative medical imaging data of a subject 401 (e.g., radiology imaging data such as MRI or CT scan) to set a location of a landmark on a video stream of an arthroscopic procedure (e.g., from video stream input 301). In some embodiments, a preoperative medical imaging (e.g., MRI) ingest module 402 interfaces with an external repository to import the preoperative medical imaging (MRI/CT) data 401. The preoperative medical imaging data may comprise radiological images of a subject. In some embodiments, the radiological images are from and associated with a joint or other boney structure of the subject, such as the shoulder, knee, hip, ankle, or spine. In various embodiments, the radiological images may be generated using one or more of fluoroscopy, magnetic resonance imaging (MRI), X-ray, computed tomography (CT) scanning, positron emission tomography (PET) scanning, or ultrasound. In some embodiments, the preoperative medical images comprise MRI or CT scan images. In some embodiments, MRI or CT scan images is acquired from the subject for an arthroscopic procedure (e.g., a knee surgery, a shoulder surgery, or a hip surgery). The MRI or CT scan images may comprise an image of a subject's knee or a shoulder. In some embodiments, the MRI, CT scan, or other images are obtained from the repository in a standard format (e.g., DICOM). In some embodiments, preoperative medical imaging ingest module 402 comprises an application programming interface (API) layer to abstract external system associated with an imaging system (e.g., MRI, CT scan, or PET imaging) from the system 400. In some embodiments, the repository of images comprises an image of a landmark. In some embodiments, the image of the landmark from the repository has been placed by an operator (e.g., a surgeon) on the MRI or CT scan images of the subject. The output from preoperative medical imaging ingest module 402 may be provided to a three dimensional (3D) image reconstruction module 403. In some embodiments, 3D image reconstruction module 403 converts volumetric image data from preoperative medical imaging ingest module 402 comprising one or more slices of two dimensional (2D) images and converts the volumetric image data into a 3D image in a computer memory. In some embodiments, the coordinates of the landmarked set by the operator are mapped onto the 3D image. In some embodiments, 3D image reconstruction module 403 may generate a multi-dimensional array comprising the 3D representation of a radiological image and the landmark mapped to the image. In some embodiments, the output from 3D image reconstruction module 403 may be merged with the mask(s) generated by the anatomy recognition module 305, using a mapping module 404. In some embodiments, mapping module 404 comprises a trained AI network to recognize anatomical structures in an image obtained preoperatively (e.g., an MRI or a CT scan image). In some embodiments, the anatomical structure may comprise a bony structure. In some embodiments, the anatomical structure may comprise a tendon. In some embodiments, the anatomical structure recognized in the image (e.g., an MRI or a CT scan image) may be masked (e.g., labeled) in mapping module 404 using the same labeling system used in anatomy recognition module 305. The anatomical structure recognized in mapping module 404 may then be matched to an anatomical structure recognized in anatomy recognition module 305. In some embodiments, the landmark specified in 3D image reconstruction module 403 may be mapped onto the anatomical structure recognized in anatomy recognition module 305. The mapping may be provided to landmark registration module 308. As described hereinbefore, landmark registration module 308 may process and send the landmark and the anatomical structure information to be overlaid onto the video stream of the surgery. In some embodiments, landmark location module 320 is adjusted for the movement of the surgical camera. In some embodiments, when a similar structure is identified from a preoperative medical image (e.g., MRI or CT scan image) and from an image from a video stream of the surgery, the two anatomical structures are matched (e.g., in MRI/visual field matching/mapping module 404), which corrects the frame for any image discrepancies associated with the surgical camera movement. In some embodiments, each frame from the video stream is corrected for the movement of the surgical camera.

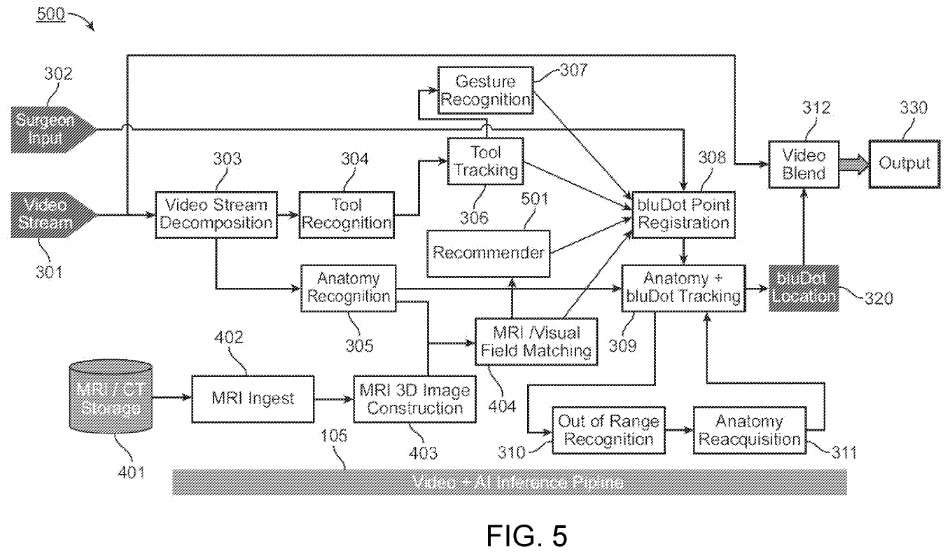

In some embodiments, the system comprises a recommender module that can recommend placement of a landmark based at least in part on the surgical context. FIG. 5 shows a schematic of an example workflow of a system to recommend a landmark placement. The system 500 shown in FIG. 5 may comprise the system 500 and a recommender module 501 to make the recommendation for placing a landmark. In some embodiments, system 500 is a surgical decision support system. In some embodiments, 501 receives anatomical feature masks or anatomical structure masks from mapping module 404. In some embodiments, based on the anatomical feature masks or anatomical structure masks received, 501 identifies a context of the surgery (e.g., anatomical region or a portal).

In some embodiments, based at least on the identified context, recommender module 501 recommends the placement of a landmark. Non-limiting examples of recommendations from 501 may comprise a femoral and tibial tunnel placement in an anterior cruciate ligament (ACL) surgery, or an anchor placement in a Rotator Cuff Repair surgery. In some embodiments, recommender module 501 may recommend the placement of a landmark based on the placement of a specialized surgical tool or probe such as a tool having a spherical and/or patterned tip as described herein. In some embodiments, recommender module 501 recommends the location of a landmark based at least in part on the location of the landmark in a preoperative medical image of the subject identified by 3D image reconstruction module 403 and/or mapping module 404. The recommended landmark and/or landmark location may be sent to landmark registration module 308 to be processed, overlaid on the video stream of the surgery, and displayed on a displaying device (e.g., a monitor), as described hereinbefore. In some embodiments, a preoperatively acquired image (e.g., MRI, CT scan, etc.) may be processed as described herein combined with tool tracking module 306 to provide the information required to estimate a size or location of a landmark. For example, imaging modalities (e.g., CT Scan, MRI, ultrasound) may produce images containing anatomical features that can be recognized by the system as described herein. The images may further comprise a location of a landmark which in various embodiments may be done with use of tool or probe positioned on the skin adjacent a target image site or within tissue of the target image site. In some embodiments, these images are three dimensional images comprising voxels, where a voxel (volumetric pixel) can represent a volume in physical space. Therefore, a location of a landmark may be identified on a surgical field of view image by matching the identified anatomical structures (e.g., by recognizing anatomical features on a preoperative image and the surgical image). The location of the landmark may be further identified based in part by measuring a size of the anatomical structure based in part on the size of the voxels in the preoperative image. This measurement may be used to place the landmark on a location on the anatomical structure on the surgical image corresponding to the location of the landmark identified on the preoperative image (e.g., CT scan or MRI).

In some embodiments, the system is configured to process a video stream from a stereoscopic surgical camera (e.g., a stereoscopic arthroscope). FIG. 6 shows a schematic flowchart of an example system to process a stereoscopic video stream (e.g., a 3D video). In some embodiments, the system 600 comprises the components in system 500 and a plurality of modules to process stereoscopic video input or stream 601. In some embodiments, the stereoscopic video input or stream 601 is first processed by a stereoscopic video decomposition module 602 to generate an image from the stereoscopic video input or stream 601. In some embodiments, stereoscopic video decomposition module 602 provides an image from the stereoscopic video input or stream 601 to a tool recognition module 603 and/or an anatomy recognition module 605. Tool recognition module 603 and anatomy recognition module 605 are similar to tool recognition module 304 and anatomy recognition module 305, respectively. In some embodiments, tool recognition module 603 and anatomy recognition module 605 are capable of processing a surgical field in an image that has a shifted view due to parallax in a stereoscopic video stream. A stereoscopic video stream or an image from the stereoscopic video stream may comprise two channels (e.g., a right side or a left side). The parallax may comprise a displacement or difference in the apparent position of an object viewed along two different lines of sight. In some embodiments, tool recognition module 603 provides one or more masks for a surgical tool to a tool localization module tool localization module 604. In some embodiments, tool recognition module 603 provide one or more masks for an anatomical structure to an anatomy localization module 606. In some embodiments, tool localization module 604 uses the differences in the perspectives of a given tool and localizes the tool in 3D space. In some embodiments, tool localization module 604 comprises tool recognition algorithms which are applied to the two channels of an image from a stereoscopic video stream (e.g., binocular video stream). A landmark may be registered using a surgical tool such a probe including various specialized probes as described herein. In some embodiments, the landmark appears in 3D space when viewed using a 3D viewing device (e.g., a binocular viewer). In some embodiments, anatomy recognition module 605 provides one or more masks for an anatomical structure to an anatomical localization module 606. In some embodiments, anatomy localization module 606 processes the anatomical structure mask(s) in the two channels of the image from a stereoscopic video stream and generates a mask that can be visualized in a 3D viewer based on the spatial information of the anatomical structure provide by anatomy recognition module 605. In some embodiments, a landmark (e.g., a colored dot, such as bluDot) is rendered to the field of view in a way that the landmark is placed in a left and a right channel of stereo display channels independently. In some embodiments, the landmark is placed with a shift (e.g., laterally) to generate depth in perception of the viewer. The output video stream 330 may comprise a landmark visualized or displayed in 3D overlaid (e.g., attached) onto an anatomical structure in a video stream of a surgery.

In some embodiments, an object (e.g., a probe or surgical tool) 801 may be placed at the same location of a landmark 802. The system may identify the tool 801 as described herein and compensate for any occlusions (FIG. 8). The landmark 802 may also move corresponding to the landmark's location as an anatomy 803 moves. FIG. 9 shows another example of the landmark 802 being cleared from an object (e.g., a tool) 801 that could block the landmark 802. FIGS. 10A-10B show the performance of the system in stabilizing a landmark 1001 (e.g., bluDot) against a movement of a camera with respect to an anatomical structure. The camera may move with respect to an anatomical structure 1002, but a landmark 1001 may remain in a marked location on the anatomical structure. In other words, the landmark 1001 may move with the anatomical structure 1002 as camera moves around.

Computer Systems

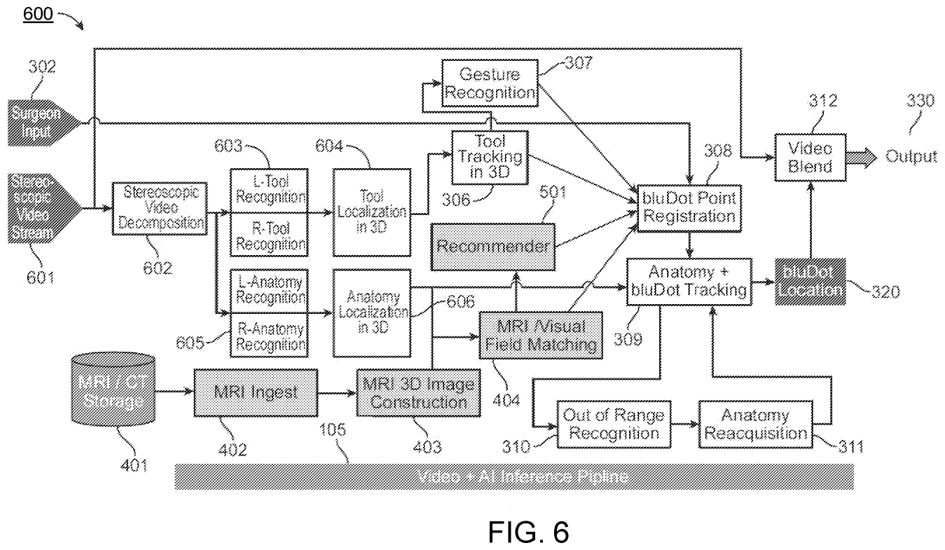

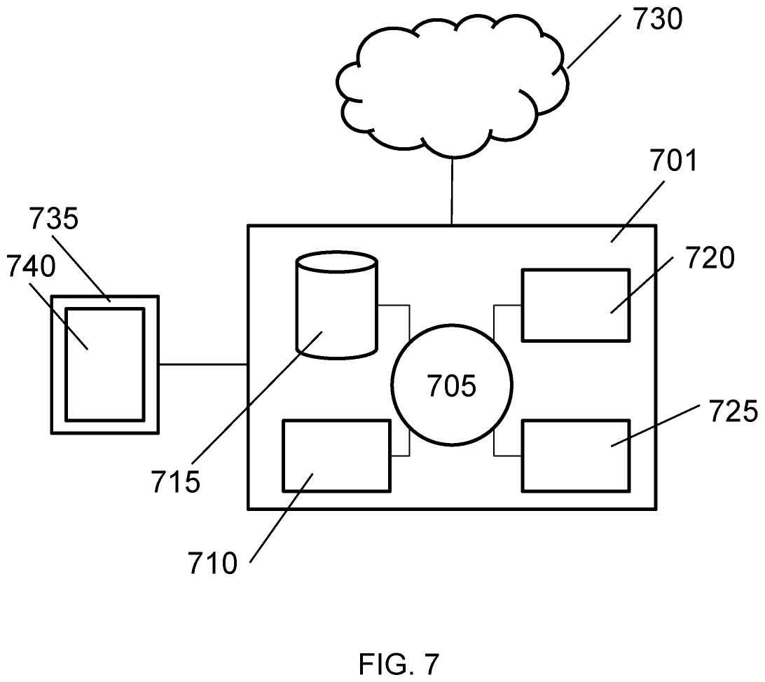

Further provided herein are computer systems that are programmed to implement methods described herein. Accordingly, a description of one or more embodiments of such computer systems will now be described. FIG. 7 shows a computer system 701 that is programmed or otherwise configured to perform one or more functions or operations of methods described herein. The computer system 701 can regulate various aspects described herein, such as, for example, of receiving an image from an interventional imaging device, identifying features in the image using an image recognition algorithm, overlaying the features on a video feed on a display device, make recommendations or suggestions to an operator based on the identified features in the image. The computer system 701 can be an electronic device of a user or a computer system that is remotely located with respect to the electronic device. The electronic device can be a mobile electronic device.

The computer system 701 includes a central processing unit (CPU, also “processor” and “computer processor” herein) 705, which can be a single core or multi core processor, or a plurality of processors for parallel processing. The computer system 701 also includes memory or memory location 710 (e.g., random-access memory, read-only memory, flash memory), electronic storage unit 715 (e.g., hard disk), communication interface 720 (e.g., network adapter) for communicating with one or more other systems, and peripheral devices 725, such as cache, other memory, data storage, and/or electronic display adapters. The memory 710, storage unit 715, interface 720, and peripheral devices 725 can be in communication with the CPU 705 through a communication bus (solid lines), such as a motherboard. The storage unit 715 can be a data storage unit (or data repository) for storing data. The computer system 701 can be operatively coupled to a computer network (“network”) 730 with the aid of the communication interface 720. The network 730 can be the Internet, an internet and/or extranet, or an intranet and/or extranet that is in communication with the Internet. The network 730 in some embodiments is a telecommunication and/or data network. The network 730 can include one or more computer servers, which can enable distributed computing, such as cloud computing. The network 730, in some embodiments with the aid of the computer system 701, can implement a peer-to-peer network, which may enable devices coupled to the computer system 701 to behave as a client or a server. Network 730 may also include or incorporate a deep learning network, such as UNet, ResNEt, and the like.

The computer system 701 can include or be in communication with an electronic display 735 that comprises a user interface (UI) 740 for providing, for example, an overlay of the identified features on a video feed from an arthroscope or to provide a recommendation to an operator in the course of a surgery. Examples of UIs include, without limitation, a graphical user interface (GUI) and web-based user interface.

The CPU 705 can execute a sequence of machine-readable instructions, which can be embodied in a program or software. The instructions may be stored in a memory location, such as the memory 710. The instructions can be directed to the CPU 705, which can subsequently program or otherwise configure the CPU 705 to implement methods described herein, such as those used to identify and generate images of a surgical probe (or other surgical tool) used in the placement of a digital landmark. Examples of operations performed by the CPU 705 can include fetch, decode, execute, and writeback. Examples of specific programing which can be executed by one or more CPUs may include or be operably coupled to a deep learning network (e.g., UNet or ResNet) to generate a segmented outline of a specialized surgical probe and also computer vision and/or shape fitting algorithms to fit the shape of the specialized surgical probe.

In one or more embodiments, CPU 705 can be part of a circuit, such as an integrated circuit. One or more other components of the system 701 can be included in the circuit. In some embodiments, the circuit is an application specific integrated circuit (ASIC).

The storage unit 715 can store files, such as drivers, libraries and saved programs. The storage unit 715 can store user data, e.g., user preferences and user programs. The computer system 701 in some embodiments can include one or more additional data storage units that are external to the computer system 701, such as located on a remote server that is in communication with the computer system 701 through an intranet or the Internet.

The computer system 701 can communicate with one or more remote computer systems through the network 730. For instance, the computer system 701 can communicate with a remote computer system of a user (e.g., a portable computer, a tablet, a smart display device, a smart tv, etc.). Examples of remote computer systems include personal computers (e.g., portable PC), slate or tablet PCs (e.g., Apple® iPad, Samsung® Galaxy Tab), telephones, Smart phones (e.g., Apple® iPhone, Android-enabled device, Blackberry®), or personal digital assistants. The user can access the computer system 701 via the network 730.

Methods as described herein can be implemented by way of machine (e.g., computer processor) executable code stored on an electronic storage location of the computer system 701, such as, for example, on the memory 710 or electronic storage unit 715. The machine executable or machine readable code can be provided in the form of software. During use, the code can be executed by the processor 705. In some embodiments, the code can be retrieved from the storage unit 715 and stored on the memory 710 for ready access by the processor 705. In some situations, the electronic storage unit 715 can be precluded, and machine-executable instructions are stored on memory 710.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 701, can be embodied in programming or other machine/electronically executable instruction sets. Various aspects of the technology may be thought of as “products” or “articles of manufacture” typically in the form of machine (or processor) executable code and/or associated data that is carried on or embodied in a type of machine readable medium. In various embodiments, machine-executable code can be stored on an electronic storage unit, such as memory (e.g., read-only memory, random-access memory, or flash memory) or a hard disk. “Storage” type media can include any or all of the tangible memory of the computers, processors, or the like, or associated modules thereof, such as various semiconductor memories, tape drives, disk drives, and the like, which may provide non-transitory storage at any time for the software programming. All or portions of the software may at times be communicated through the Internet or various other telecommunication networks (including wireless and wired networks). Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

Specialized Surgical Tools or Probes for Facilitating Placement of a Virtual Landmark or Fiducial.

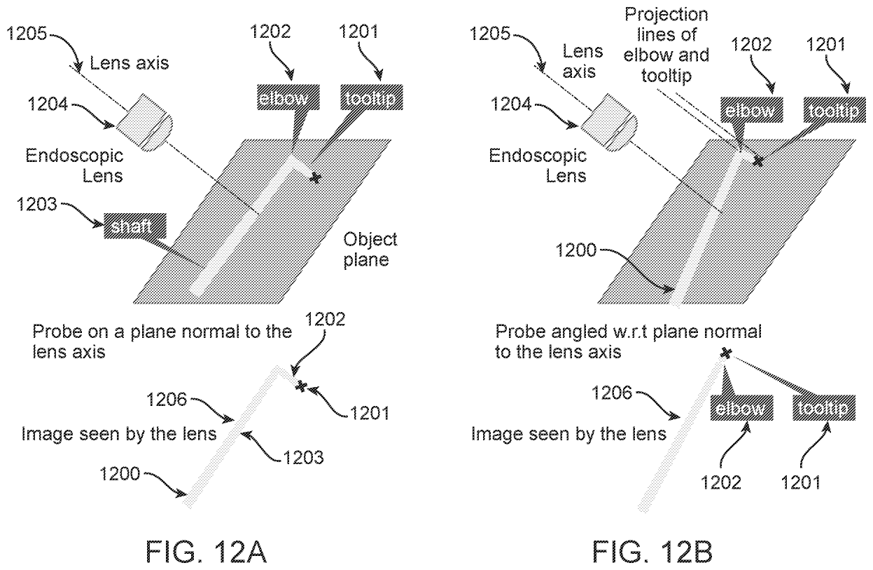

In the course of using embodiments of the algorithms described herein to perform measurements and place landmarks, the surgeon or other medical professional may elect to use surgical probes or other suitable surgical tools to indicate or highlight anatomical landmarks of interest. Various embodiments of the algorithms described herein, such as tool recognition and tracking algorithms, may be configured to process and otherwise utilize the images of the probes in the field of view of an arthroscope video camera (or other medical imaging device using during the particular surgical/medical procedure) to place digital landmarks and perform measurements in an intraoperative setting. The accuracy of the measurements and the stability of the digital landmarks may be influenced by the accuracy of the outlines of the probe as discerned by the tool recognition and tool tracking modules. However, the accuracy of those outlines may be adversely affected depending upon one or more of the size and shape of the probe including the geometry or shape of the probe tip, the orientation, and/or viewing angle of the probe with respect to arthroscopic camera (or other medical imaging camera) as well as the lighting/illumination conditions of the probe by the light source on the arthroscope or other imaging device. Various embodiments described herein present solutions to these and related problems through the use of specialized probes (or other surgical tools), specialized algorithms, and algorithm hierarchies. In particular embodiments, the specialized algorithms are designed to work with specialized probes having geometries that minimize the error and/or uncertainty in the location and placement of a virtual (e.g., a digital) landmark in the form a colored marking, such as a blue dot. With reference to FIGS. 12A-17C, further details of embodiments of the specialized probes and algorithms and/or modules for recognizing probes and facilitating placement of a virtual landmark will now be described.

FIGS. 12A-12B show perspective views illustrating the impact of tool angle on embodiments of algorithms for the recognition of surgical tools used in the operating field. In FIG. 12A, a probe 1200 having tooltip 1201, elbow 1202, and shaft 1203, is viewed by a system described herein (e.g., systems 300, 400, 500, etc.) through and endoscopic (or other imaging device) lens 1204 via lens axis 1205 such that the probe is on a plane normal to the lens axis 1205. The resulting image of the probe 1206 shows tooltip 1201, elbow 1202, and shaft 120. In FIG. 12B, the probe is angled with respect to the plane normal to the lens axis 1205 such that parts of the probe are occluded in the resulting image of the probe 1206. As a result, the system may identify the probe and compensate for any occlusions.

Further examples of probe position relative to the lens axis of the imaging device and corresponding compensation for perception of the tool tip and the subsequent landmark determination provided by systems and methods described herein are summarized in TABLE 1.

TABLE 1

| Probe Position | Compensation |
|---|---|
| Probe is on a plane normal | Landmark location determination is |
| to the axis of the lens. | relatively simple; minimal compensation |
| | is needed to estimate the width of the tip. |
| Probe tip is on a plane | Landmark location determination is |
| normal to the axis of the lens. | relatively simple; moderate is needed |
| | to estimate the width of the tip. |
| Axis of the probe is angled | Heavy computation needed to estimate |
| upwards towards the lens, | the intended landmark location; |
| but the tip is angled away | significant compensation needed to |
| from the lens; the elbow of | estimate the width of the tip. |
| the 90 degree bend is visible. | |
| Axis of the probe is either | Landmark location determination is |
| normal to the axis of the | relatively simple, but has some |
| lens or angled away from the | uncertainty; minimal compensation |
| lens; the elbow of the 90 | is needed to estimate the width of |
| degree bend is not visible. | the tip, but has some uncertainty. |

Embodiments of Probes Having a Spherical Tip

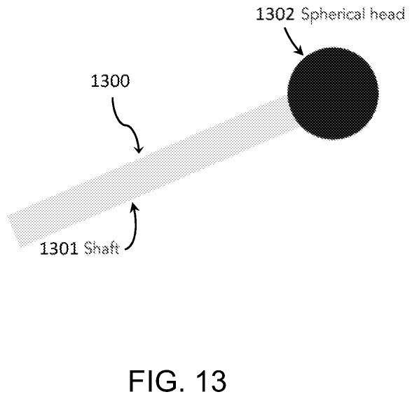

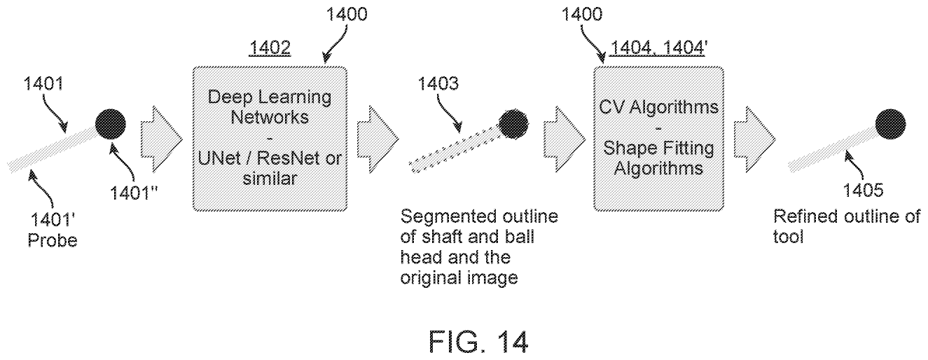

According to some embodiments a specialized probe (or other surgical tool) for facilitating location and placement of digital landmarks include a shaft and a spherical tip or other rounded geometry. FIG. 13 illustrates an embodiment of one such specialized probe 1300 having a shaft 1301 and a spherical tip 1302. Embodiments of such a tip may provide several advantages and solutions to the problem of error or inaccuracy in recognition and/or rendering of a pixel mask of the probe and/or more refined outline of the probe including the tip. A key advantage is that a spherical tip appears as a circle from any viewing angle of the imaging device camera. Therefore, the embodiments of the tool recognition and tool tracking modules may need to compensate only for depth along the axis of the lens. For example, CV algorithms can be used to tighten the outlines of the sphere after initial segmentation is performed. Another advantage is that the spherical tip permits post processing to obtain a more accurate outline of the tooltip in instances where the tool recognition module outputs a mask which is of low confidence. In various embodiments, the spherical tip can be constructed to have a selected (or known) size (e.g., diameter) with a selected accuracy (e.g., ±5%, ±2.5%, or ±1%) and degree of sphericity (e.g., 0.9, 0.95, or 0.98). The size of the probe can then be manually or automatically entered into the tool recognition module so as to improve the accuracy of the refined outline of the probe, and in turn, the placement of the digital landmark. Probes with greater degrees of accuracy and sphericity may further improve these outcomes. According to some embodiments, the probe may have optical indicia or other indicia (e.g., on the tip or shaft) indicating the size, accuracy, and sphericity of the probe tip, which can be detected by an imaging device used during the procedure (e.g., an arthroscopic). that the optical indicia or other indicia information may then be processed and inputted into the tool recognition module (such as that shown in FIGS. 14 and/or 15) or other related module or algorithm. In use, such embodiments may allow the surgeon or other medical practitioner to automatically enter the dimension of the probe tip into one or more modules for recognizing the probe/tool, as well as mapping the target point of the probe. In alternative embodiments, the probe tip can be configured to be expandable/deployable to preselected dimensions (e.g., diameter) so as to allow for surgical situations requiring different sized probes (e.g., when the probe is moved from one anatomical location to another). An expandable/deployable probe tip also may allow for multiple target point computations to be performed with different sized probe tips to improve accuracy by compensating for various factors which may introduce errors. These factors may include distance from the camera and situations where portions of the probe may be obstructed from the field of view of the camera and/or is insufficiently illuminated (e.g., by arthroscopic light source). In various embodiments, the probe or tool tip can have a texture (e.g., a matte texture) which can be configured to eliminate or reduce reflections of the tip that can obscure detection or recognition by one or embodiments of systems described herein (e.g., systems 300, 400, 500, etc.). In additional or alternative embodiments, the tip can be also colored for maximal contrast with the background (e.g., surrounding tissue).

Embodiments of Alternative Probe Designs

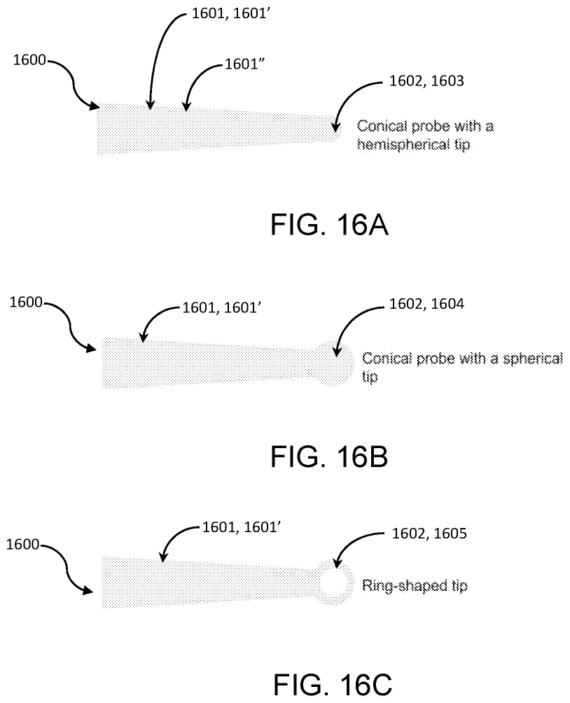

Referring now FIGS. 16A-16C, in various embodiments, the shaft 1601 of tool 1600 may have a conical shape 1601′ configured to facilitate recognition by a deep learning network (e.g., network 1402), a machine learning algorithm, and/or a computer vision algorithm (1404) executed on the one or more processors that are part of embodiments of systems described herein (e.g., system 300, 400, 500, and 701) for location and placement of a digital landmark or operatively coupled to the system. In these and related embodiments, the shape of the probe or tool tip 1602 may be hemispherical, spherical, or annular. FIG. 16A shows a probe 1600 having conical shaft 1601 and a hemispherical tip 1603. The conical edge 1600″ of the probe 1600 can be detected robustly using algorithms described herein. For example, using a CV component algorithm such as 1404, the edges can be extrapolated to the center of the hemispherical tip 1603 to provide precise estimates of both the shaft edge 1600″ and the tip location. FIG. 16B shows a conical probe 1600 (e.g., a probe having conical-shaped shaft 1601′) with a spherical tip 1604. Using a CV component (e.g., module 1404), the edges can be extrapolated to the center of the spherical tip to provide precise estimates. The spherical tip 1604 can provide an additional level of fault-tolerance to the estimates. FIG. 16C shows a conical probe 1600 with a ring-shaped tip 1605. Using a CV component (e.g., module 1404), the edges can be extrapolated to the center of the ring tip to provide precise estimates. The ring-shaped tip 1605 can provide visual feedback to the surgeon about precisely where the landmark is being placed.

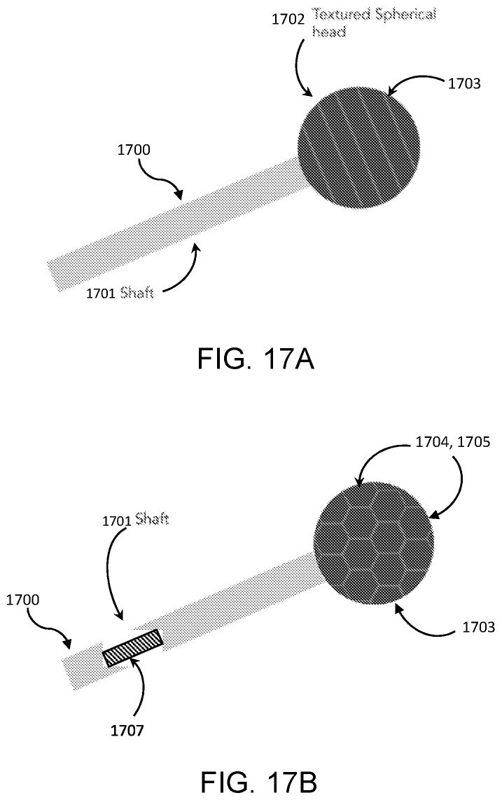

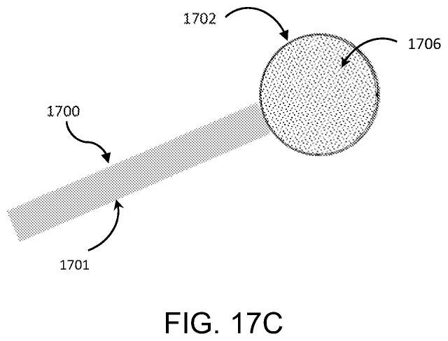

FIGS. 17A-17C show alternative or additional embodiments of probes or other tools having specialized patterns, textures, or contrast to facilitate recognition by algorithms described herein. These example probes include a shaft 1701 and a head or tip 1702 having a pattern 1703 or texture 1706. Head or tip 1702 typically will be spherical in shape; however, other geometries are also considered, including hemispherical or ring shaped. Suitable patterns 1703 for head 1702 can include lined or cross hatched (as shown in FIG. 17A) and crisscross, as well as a variety of geometric patterns 1704, including for example various polygons 1705 (as shown in in FIG. 17B), such as one or more of a pentagon, hexagon, octagon and the like. In use, embodiments of patterns 1703 including geometric patterns 1704 for tool head or tip 1702, can allow for the entire shape of the tip to be recognized by an arthroscope or imaging device even when only a portion is visible to the imaging device by reconstruction of the obscured portions of the tip based on the patterned portion that can be viewed. The reconstruction can be performed by a deep learning network, machine learning algorithm, or computer vision algorithm executed on one or more processors that are part of embodiments of system described herein (e.g., systems 300, 400, 500, 701, etc.) or operatively coupled to embodiments of such systems. The algorithms or learning networks can be trained to recognize the patterns on the probe. In alternative embodiments, a particular probe tip pattern 1703 can be inputted into one or more of the algorithms or modules described herein such as tool recognition module 1400 described below. In related alternative embodiments, probe or tool 1700 may include optical indicia 1707 encoding information on a particular geometric pattern 1703 which can be detected by the medical imaging device and then read by the algorithm and/or deep learning network. Indicia 1707 may also encode information on the shape, dimensions and size of the probe or tool including that of the probe/tool tip or head. In various embodiments, indicia 1707 may correspond to a bar code, a QR code, or other machine-readable optical marking encoding information. Indicia 1707 will typically be placed on tool shaft 1701, but alternatively, can be placed on or near tip or head 1703.

With specific reference to FIG. 17C, in various embodiments, the probe or tool tip 1703 can have a texture 1706 (e.g., a matte texture) which can be configured to eliminate or reduce reflections of the tip that can obscure detection or recognition by one or embodiments of systems described herein (e.g., systems 300, 400, 500, etc.). In additional or alternative embodiments, the probe or tool tip 1702 can be also colored for maximal contrast with the tissue site background (e.g., surrounding tissue) where the probe or tool is used. Suitable contrasting colors including green for a red background, and black for a white light grey background.

Embodiments of Tool Recognition Algorithms for Specialized Probes

Referring now to FIG. 14, an embodiment of a tool recognition module 1400 for use with embodiments of one or more specialized probes (or other surgical tools) will now be described. This and related embodiments of tool recognition module 1400 can be configured and used to obtain a high-confidence and precise outline of the probe (or other surgical tool) including the probe tip for use in facilitating the location and placement of embodiments of a digital landmark described herein. An image 1401 of a probe (or other tool) 1401′ from an endoscope/arthroscope (or other imaging device) can be supplied to the first component 1402 of module 1400. This component can include or correspond to a suitably trained deep learning network, e.g., a deep learning network which may include UNet, ResNet or other neural network architecture known in the art. For ease of discussion, component 1402 will now be referred to as a network 1402. The network 1402 can then output a segmented image 1403 depicting the pixels where the probe or tool 1401′ is detected or recognized. This image can be operated on (e.g., processed) by a computer vision algorithm or module 1404, which may include or be operatively coupled to a shape fitting algorithm 1404′. Algorithms 1404 and 1404′ which may correspond to those described herein or known in the art can be used to fit the most appropriate shape, i.e., a shape which minimizes the error onto the pixel mask. For embodiments of the tool (e.g., probe) 1401′ where the tool tip 1401″ is spherical, the tooltip appears as a circle regardless of the angle at which the axis of the tool is held. CV algorithms can be used to find a best fit circle, thereby generating a refined outline 1405 of the tool. This refined outline of the tool can be used in downstream processing.

In various embodiments, shape fitting algorithm 1404′ can also be configured and used to improve detection and depiction of the tool tip 1401″ in intraoperative or other clinical settings where visualization of the tool tip is obscured or lighting conditions in the field of view of the arthroscopic or other imaging device are less than optimal. For example, in intraoperative settings for minimally invasive procedures, surgeons generally utilize a one point source of light coming from the arthroscope, endoscope or other imaging device. This arrangement can cause the outline of the tooltip to be less well defined, e.g., if the angle of light incident on the tooltip is suboptimal. When the lighting is suboptimal, the confidence with which the pixels along the outline of the tool are detected can be lower. In some embodiments, a shape fitting algorithm component such as that in algorithm 1404′ can be applied to improve the confidence scores of the pixels. Portions of the outlines with higher confidence pixels can be used to complete the shape, thereby improving the detection of the tooltip under poor lighting conditions.

Algorithm and Learning Network Training and Configuration Modes

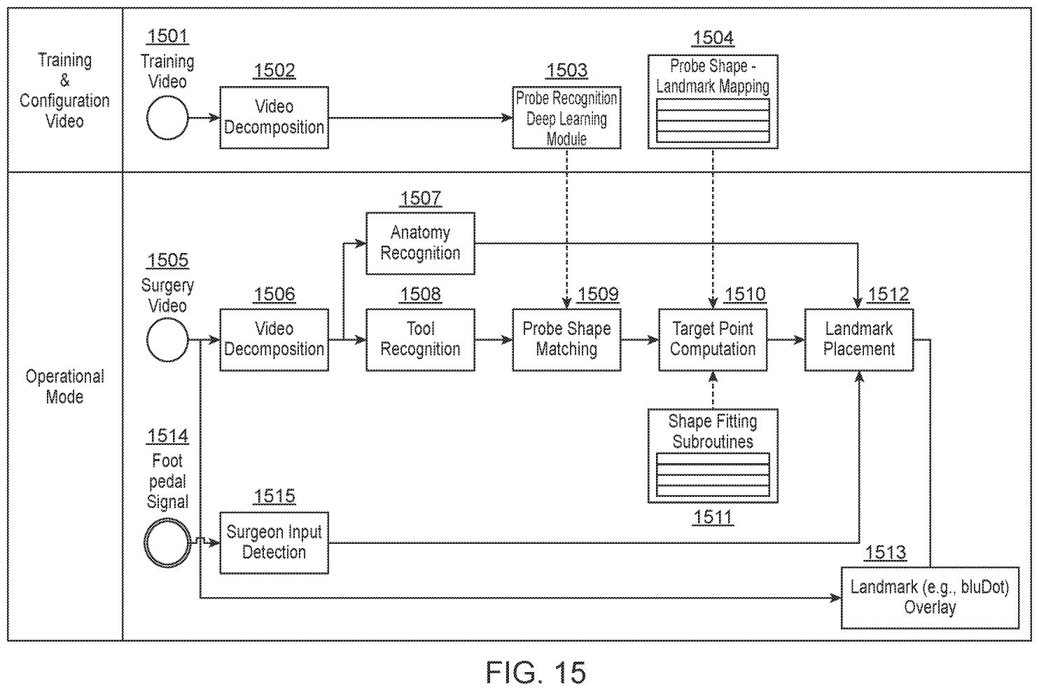

Referring now to FIG. 15, in various embodiments, one or more algorithms or learning networks described herein including those used for tool recognition (e.g., network 1402 or algorithm 1404) and digital landmark location and placement may be configured to operate in a Training and Configuration Mode and an Operational Mode. FIG. 15 is a flow chart illustrating a method described herein utilizing both modes and the interplay between the two modes.

Training and Configuration Mode

According to one or more embodiments of the invention, when operating in the training and configuration mode, the Probe Recognition Deep Learning Module can be supplied with video streams 1501 of specialized surgical probes moving around against a plain background with minimal specular reflections. The probes can be handled by a subject matter expert; the movements performed can reflect the motions a surgeon would perform when using the probe during surgery. This video stream can be decomposed into individual frames via the video decomposition module 1502, and then, CV techniques can be used to isolate the tool from the background. These images can then be used to train an image recognition module 1503 (e.g., for recognizing an image of a surgical probe or other surgical tool) using a deep learning network, such as UNET operating on a ResNET backbone. This process can be repeated with each surgical probe from a catalog of supported devices. In this manner, the network can gain the ability to recognize all the devices in the surgical catalog.

A separate configuration module 1504 can match the probes with the target point of the probe (e.g., the location where the digital landmark is to be placed). The configuration module 1504 can be used to create a mapping between each probe and the point on or off the probe where a surgeon intends to place a digital or other virtual landmark. For example, if the probe has a conical point, the target point can be assigned to the tip of the cone. If the probe has a spherical point, the target point can be assigned to the projection of the center of the sphere on the underlying anatomical structure.

Operational Mode

According to one or more embodiments of the invention, when operating in the operational mode, a video stream 1505 of a surgery or other medical procedure from an imaging device can be decomposed into individual frames via the video decomposition module 1502. The frames can then be supplied to the anatomy recognition module 1507 and the tool recognition module 1508. The tool recognition module 1508 can implement a deep learning algorithm, e.g., UNet over a ResNET backbone. The pixel-mask with the outline of the mask can be supplied to the probe shape matching module 1509. This module can use the neural networks built during the training mode (e.g., the probe recognition deep learning module 1503) to find the closest match to the probe observed in the surgical field of view. Once the closest match is identified, the mapping between the shape of the probe and the intended location of the landmark can be obtained from the configuration module 1504. Module 1504 or another module (e.g., target point computation module 1510) can activate a subroutine, e.g., shape fitting subroutine 1511, based on the mapping. For example, if the shape of the probe most closely matches a sphere, the shape fitting subroutine 1511 can refine the outline of the mask obtained from the tool recognition algorithm 1503. CV algorithms can be used to generate a circle which most closely fits the obtained outlines. Once the fitted circle is obtained, the center of the circle can be computed via a target point computation module 1510. This target point can then be passed along to the landmark placement module 1512. If the shape of the probe most closely matches a cone, a different subroutine can first determine the edges of the cone by fitting two straight lines along the tapering edges of the cone. The outline thus created can be robust against the pixel-level errors along the outlines of the edge. The tip can be determined by extending the two lines and computing the point in space where the lines intersect. In the absence of this technique, the uncertainty involved in determining the point would be high. This point can be passed along to the landmark placement module 1512 to display a landmark overlay via landmark overlay module 1513.

Also, in various embodiments of the operational mode, an operator (e.g., a surgeon) can manually provide one or more sets of coordinates of a landmark, for example, via a foot pedal signal 1514. The signal 1514 can be received by the surgeon (or other operator) input detection algorithm 1515. Landmark placement can then be performed by landmark placement module 1512. A resulting landmark overlay can then be displayed via landmark overlay module 1513.

### CONCLUSION

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. Accordingly, it should be understood that the invention covers various alternatives, modifications, variations or equivalents to the embodiments of the invention described herein.

Also, elements, characteristics, or acts from one embodiment can be readily recombined or substituted with one or more elements, characteristics or acts from other embodiments to form numerous additional embodiments within the scope of the invention. Moreover, elements that are shown or described as being combined with other elements, can, in various embodiments, exist as standalone elements. It is contemplated that such elements can include an algorithm or portion of an algorithm (e.g., a subroutine) as well as a dataset (e.g., a training data set) or portion of a data set. Further, embodiments of the invention specifically contemplate the exclusion of an element, act, or characteristic, etc. when that element, act or characteristic is positively recited. Hence, the scope of the present invention is not limited to the specifics of the described embodiments but is instead limited solely by the appended claims.

1. A system for performing tissue landmarking during a medical procedure in a patient, the landmarking performed with a tool having a tool tip with a known geometry and size, the system comprising:
one or more processors;
a memory to store instructions operable on the one or more processors; and
an interface to receive a video stream encoding image data from an imaging device positioned to image a scene of the tool positioned at a selected tissue location of the patient, wherein the one or more processors execute instructions stored in the memory to process the image data received from the interface and to perform operations comprising:
a) recognizing the tool from the image data and generating a segmented tool outline depicting pixels where the tool is detected, wherein the segmented tool outline is represented as a pixel mask;
b) fitting a shape of the tool onto the pixel mask by utilizing the geometry and size of the tool tip, wherein the fitted shape of the tool minimizes error or inaccuracy of a shape of the pixel mask;
c) mapping a location to be landmarked based on the geometry and size of the tool tip;
d) utilizing the fitted shape of the tool and the mapped location to generate a digital landmark at the selected tissue location; and
e) overlaying the digital landmark onto the video stream.

2. The system of claim 1, wherein the scene is that of a selected tissue site or intraoperative tissue site.

3. The system of claim 1, wherein the segmented tool outline depicts only pixels where the tool is detected.

4. (canceled)

5. The system of claim 1, wherein the error or inaccuracy of the shape of the pixel mask is caused by a viewing angle of the imaging device relative to the tool or the tool tip or by suboptimal lighting of the tool or the tool tip.

6-7. (canceled)

8. The system of claim 1, wherein the mapping of the location to be landmarked is further based on a predetermined target point of the tool tip.

9. The system of claim 8, wherein the predetermined target point is determined using a machine learning algorithm dataset, a training data set, or a combination thereof.

10. The system of claim 1, wherein the imaging device is an arthroscope, an endoscope or a laparoscope.

11. (canceled)

12. The system of claim 1, wherein the recognition of the tool or the generation of the segmented tool outline is performed using a deep learning network or architecture.

13. (canceled)

14. The system of claim 1, wherein fitting of the shape of the tool onto the pixel mask is performed using a computer vision algorithm or a shape fitting algorithm.

15-17. (canceled)

18. The system of claim 1, further comprising the tool.

19. The system of claim 18, wherein the tool comprises a surgical probe.

20. The system of claim 18, wherein the tool tip has a rounded geometry or a spherical geometry.

21. (canceled)

22. The system of claim 20, wherein the tool tip has a pattern, a texture, or a contrast configured to enhance recognition of the tool tip by a deep learning network, a machine learning algorithm, or a computer vision algorithm executed on the one or more processors.

23-25. (canceled)

26. The system of claim 1, wherein the tool comprises a shaft having a conical shape configured to have enhanced recognition of the shaft by a deep learning network, a machine learning algorithm, or a computer vision algorithm executed on the one or more processors.

27. The system of claim 26, wherein the tool tip has a spherical, hemispherical, or annular geometry.

28. A system for performing tissue landmarking during a medical procedure in a patient, the landmarking performed with a tool having a tool tip with a known geometry and size, the system comprising:
one or more processors;
a memory to store instructions operable on the one or more processors;
an interface to receive a video stream encoding image data from an imaging device positioned to image a scene of the tool positioned at a selected tissue location of the patient, wherein the one or more processors execute instructions stored in the memory to process the
image data received from the interface and to perform operations comprising:
a) recognizing the tool from the image data and generating a segmented tool outline, wherein the segmented tool outline comprises a pixel mask;
b) fitting a shape of the tool onto the pixel mask by utilizing the geometry and size of the tool tip, wherein the fitted shape of the tool minimizes error or inaccuracy of a shape of the pixel mask;
c) mapping a location to be landmarked based on the geometry and size of the tool tip;
d) utilizing the fitted shape of the tool and the mapped location to generate a digital landmark at the selected tissue location; and
e) overlaying the digital landmark onto the video stream; and
the tool, wherein the tool comprises a shaft and the tool tip is coupled to the shaft, wherein the tool tip is a patterned tool tip configured to enhance recognition of the tool tip by a deep learning network, a machine learning algorithm, or a computer vision algorithm executed on the one or more processors.

29. The system of claim 28, wherein the error or inaccuracy of the shape of the pixel mask is caused by a viewing angle of the imaging device relative to the tool or the tool tip.

30-31. (canceled)

32. A computer-implemented method for performing tissue landmarking during a medical procedure in a patient, the landmarking performed with a tool having a tool tip with a known geometry and size, the method comprising:
receiving a video stream from an imaging device, wherein the video stream encodes image data;
analyzing the image data and recognizing the tool from the image data;
generating a segmented tool outline depicting pixels where the tool is detected, wherein the segmented tool outline is represented as a pixel mask;
fitting a shape of the tool onto the pixel mask by utilizing the geometry and size of the tool tip, wherein the fitted shape of the tool minimizes error or inaccuracy of a shape of the pixel mask;
mapping a location to be landmarked based on the geometry and size of the tool tip;
utilizing the fitted tool shape and mapped location to generate a digital landmark at a selected tissue location where the tool tip is positioned; and
overlaying the digital landmark onto the video stream, thereby generating an overlaid video stream.

33. The method of claim 32, wherein the segmented tool outline depicts only pixels where the tool is detected.

34-36. (canceled)

37. The method of claim 32, further comprising displaying the overlaid video stream on one or more display devices.

38-67. (canceled)
